(19) United States
(12) Patent Application Publication
(10) Pub. No.: US 2004/0266848 A1
(43) Pub. Date: Dec. 30, 2004

(54) Preparation of N2-alkylated 1,2,3-triazoles

(76) Inventors: Mark Allen Hadd, Ann Arbor, MI (US); Brian Joseph Nichelson, Hudson, MI (US); Zhijian Zhu, Farmington Hills, MI (US)

Correspondence Address: WARNER-LAMBERT COMPANY, 2800 PLYMOUTH RD, ANN ARBOR, MI, 48105, US

(21) Appl. No.: 10/876,890

(22) Filed: Jun. 25, 2004

Related U.S. Application Data
(60) Provisional application No. 60/483,432, filed on Jun. 27, 2003.

(51) Int. Cl. A61K 31/422; C07D 413/02

(52) U.S. Cl. 514/374; 548/235

(57) ABSTRACT

Methods and materials for preparing N2-alkylated triazoles, such as 3-{4-[3-(5-methyl-2-phenyl-oxazol-4-yl)-propyl]-phenyl}-2-[1,2,3]triazol-2-yl-propionic acid, are disclosed. Such compounds are PPAR agonists that are useful for treating non-insulin dependent diabetes.

### CROSS REFERENCE TO RELATED APPLICATION

This application claims the benefit of U.S. Provisional Patent Application No. 60/483,432, filed Jun. 27, 2003.

### BACKGROUND OF THE INVENTION

1. Field of Invention

This invention relates to materials and methods for preparing N2-alkylated triazoles, such as 3-{4-[3-(5-methyl-2-phenyl-oxazol-4-yl)-propyl]-phenyl}-2-[1,2,3]triazol-2-yl-propionic acid, which are PPAR agonists useful for treating non-insulin dependent diabetes.

2. Discussion

Certain N2-alkylated triazoles (see Formula 1 below) have been shown to stimulate one or more peroxisome proliferator-activated receptors (PPARs). See commonly assigned International Patent Application WO 03/018553 A1 (the '553 Application), published Mar. 6, 2003, which is herein incorporated by reference in its entirety for all purposes. These receptors are members of the nuclear receptor superfamily of transcription factors, which includes steroid, thyroid, and Vitamin D receptors. PPARs play an important role in controlling expression of proteins that regulate lipid metabolism, and include three subtypes—PPAR α, PPAR δ, and PPAR γ—each displaying a different pattern of tissue expression and activation.

For example, PPAR γ is expressed most abundantly in adipose tissue and at lower levels in skeletal muscle, heart, liver, intestine, kidney, vascular endothelial, and smooth muscle cells, and mediates adipocyte signaling, lipid storage, and fat metabolism. Recent data support the conclusion that PPAR γ is the primary, and perhaps the exclusive, molecular target mediating insulin-sensitizing action of one class of antidiabetic agents—thiazolidine 2,4 diones. This and other data suggest that PPAR γ agonists should prove useful in treating non-insulin dependent diabetes. Indeed, recent studies indicate that 3-{4-[3-(5-methyl-2-phenyl-oxazol-4-yl)-propyl]-phenyl}-2-[1,2,3]triazol-2-yl-propionic acid and structurally related compounds are potentially potent antidiabetic agents. See, e.g., the '553 Application.

The '553 Application describes various methods of making compounds of Formula 1. One useful approach is exemplified by the preparation of 3-{4-[3-(5-methyl-2-phenyl-oxazol-4-yl)-propyl]-phenyl}-2-[1,2,3]triazol-2-yl-propionic acid. The method includes successive alkylations in which [1,2,3]triazole is first reacted with ethyl bromoacetate to give a desired N2 isomer, [1,2,3]triazol-2-yl-acetic acid ethyl ester, which is subsequently reacted with p-bromobenzylbromide (p-BBB) to give 3-(4-bromo-phenyl)-2-[1,2,3]triazol-2-yl-propionic acid ethyl ester. The method also employs a palladium-catalyzed cross-coupling reaction between the bromobenzyl triazole and 9-(5-methyl-2-phenyl-oxazol-4-yl-propyl)-9-bora-bicyclo[3.3.1]nonane (9-BBN), followed by base-catalyzed hydrolysis of the ester function to generate the final product.

Though useful, the method presents challenges. For example, the predominant products of the first and second alkylations are, respectively, an N1 isomer, [1,2,3]triazol-1-yl-acetic acid ethyl ester, and a bis-alkylated compound, 2-(4-bromo-benzyl)-3-(4-bromo-phenyl)-2-[1,2,3]triazol-2-yl-propionic acid ethyl ester. The preferential formation of the N1 isomer and the bis-alkylation product results in relatively modest yields of the desired N2 isomer and bromobenzyl product (22% and 26%), which together with yield losses from the cross-coupling and hydrolysis reactions, results in an overall yield of about 3.5%. Additionally, the method relies on numerous chromatographic separations, which make the process problematic for commercial scale-up. Thus, other methods are needed to prepare compounds of Formula 1.

### SUMMARY OF THE INVENTION

The present invention provides materials and methods for preparing compounds of Formula 1 and Formula 10, including pharmaceutically acceptable salts, esters, amides, and prodrugs thereof. The claimed method avoids the use of multiple chromatographic separations and provides significant yield improvements when compared to other methods. It is particularly advantageous for preparing 3-{4-[3-(5-methyl-2-phenyl-oxazol-4-yl)-propyl]-phenyl}-2-[1,2,3]triazol-2-yl-propionic acid and structurally related compounds, which are known mixed PPAR α/γ agonists and potentially potent agents for treating non-insulin dependent diabetes. As indicated below, the method exhibits an overall yield of about 37% when used to prepare 3-{4-[3-(5-methyl-2-phenyl-oxazol-4-yl)-propyl]-phenyl}-2-[1,2,3]triazol-2-yl-propionic acid.

Therefore, one aspect of the present invention provides a method of making a compound of Formula 1,

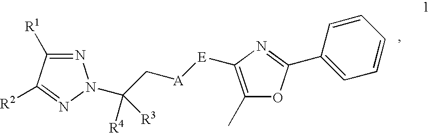

in which R¹ and R² are independently hydrogen, halogen, aryl, benzoyl, C₁₋₆ alkyl, C₁₋₆ haloalkyl, C₁₋₆ alkanoyl, C₁₋₆ haloalkanoyl, or C₃₋₇ cycloalkanoyl;

R³ and R⁴ are electron-withdrawing groups, which may be the same or different;

E is C₁₋₆ alkyleneoxy, C₁₋₆ alkyleneamino, C₁₋₆ alkylenethio, C₁₋₆ alkanediyl, C₁₋₆ alkenediyl, or C₁₋₆ alkyndiyl; and

A is arylene (including phenylene) or heteroarylene, each of which may have one or more non-hydrogen substituents, provided that when A is a five-member heteroarylene group, A is not linked to E through a heteroatom.

The method includes reacting a [1,2,3]triazole salt of Formula 2,

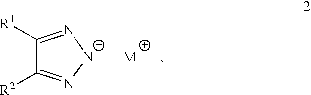

with a compound of Formula 3,

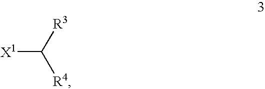

to yield a compound of Formula 4,

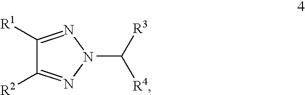

wherein R¹, R², R³, and R⁴ are as defined above for Formula 1, M is a counter ion, and X¹ is a leaving group. The [1,2,3]triazole salt of Formula 2 may be prepared in situ. The method also includes reacting the compound of Formula 4 with a compound of Formula 7,

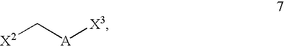

to yield a compound of Formula 8,

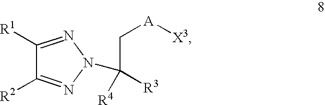

wherein R¹, R², R³, R⁴, and A are as defined above for Formula 1, X² is a leaving group, and X³ is a leaving group or a nucleophilic group, which may include hydroxy, amino, or thio. The compound of Formula 8 is subsequently coupled with a compound of Formula 9,

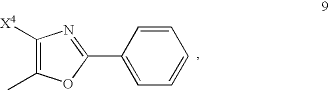

to yield the compound of Formula 1. In Formula 9, X⁴ is a C₁₋₆ hydroxyalkyl, C₁₋₆ oxoalkyl, C₁₋₆ haloalkyl, C₂₋₆ alkenyl, or C₂₋₆ alkynyl. The method optionally includes converting the compound of Formula 1 into a pharmaceutically acceptable salt, ester, amide, or prodrug. The method may also include removing R³ (or R⁴) to yield a compound of Formula 10,

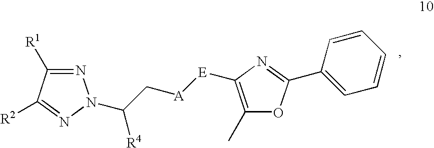

(or its analog) and if desired, converting the resulting compound into a pharmaceutically acceptable salt, ester, amide, or prodrug.

The method may further include reacting the [1,2,3]triazole salt of Formula 2 with the compound of Formula 3 to yield a mixture of N-alkylated triazoles, which includes the compound of Formula 4 and at least one compound of Formula 5,

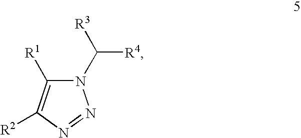

wherein R¹, R² R³, and R⁴ are as defined in Formula 1; reacting the at least one compound of Formula 5 with an alkylating agent to yield one or more N1,N3-bisalkylated triazolium intermediates; and precipitating out of solution the one or more N1,N3-bisalkylated triazolium intermediates through contact with a solvent.

The method may also include reacting the [1,2,3]triazole salt of Formula 2 with the compound of Formula 3 to yield a mixture of N-alkylated triazoles, which includes the compound of Formula 4 and at least one compound of Formula 5; reacting the mixture of N-alkylated triazoles with a compound of Formula 7, to yield a mixture comprised of the compound of Formula 8 and at least one compound of Formula 14,

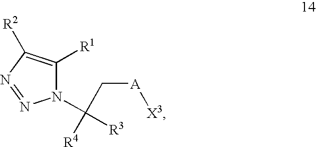

wherein R¹, R², R³, R⁴, A, and X³ are as defined above in connection with Formula 1 and Formula 7; reacting the at least one compound of Formula 14 with an alkylating agent to yield one or more N1,N3-bisalkylated triazolium intermediates; and precipitating out of solution the one or more N1,N3-bisalkylated triazolium intermediates through contact with a solvent.

Another aspect of the present invention provides a method of making a compound of Formula 4, and includes reacting the [1,2,3]triazole salt of Formula 2 with a compound of Formula 3 to yield the compound of Formula 4, where Formula 2, Formula 3, and Formula 4 are given above.

An additional aspect of the present invention provides a method of concentrating or enriching an N2-alkylated triazole of Formula 4 or Formula 8, in a mixture of N-alkylated triazoles that includes at least one N1-alkylated triazole of Formula 5 or Formula 14, respectively. The method includes reacting the mixture of N-alkylated triazoles with an alkylating agent to convert the at least one N1-alkylated triazole to one or more N1,N3-bisalkylated triazolium intermediates. The method also includes contacting the one or more N1,N3-bisalkylated triazolium intermediates with a solvent that is adapted to precipitate out of solution the one or more N1,N3-bisalkylated triazolium intermediates while leaving the N2-alkylated triazole in solution, where Formula 4, Formula 5, Formula 8, and Formula 14 are shown above.

A further aspect of the present invention provides compounds of Formula 4 or Formula 8, as shown above, including salts thereof, in which

R¹ and R² are independently hydrogen, halogen, aryl, benzoyl, C₁₋₆ alkyl, C₁₋₆ haloalkyl, C₁₋₆ alkanoyl, C₁₋₆ haloalkanoyl, or C₃₋₇ cycloalkanoyl;

R³ and R⁴ are each an electron-withdrawing group, which may be the same or different provided that R³ and R⁴ are not both methoxycarbonyl or ethoxycarbonyl;

A is arylene or heteroarylene, each of which may have one or more non-hydrogen substituents; and

X³ is a leaving group or a nucleophilic group, including hydroxy, amino, or thio.

### DETAILED DESCRIPTION

Definitions and Abbreviations

Unless otherwise indicated, this disclosure uses definitions provided below. Some of the definitions and formulae may include a “-” (dash) to indicate a bond between atoms or a point of attachment to a named or unnamed atom or group of atoms. Other definitions and formulae may include an “=” to indicate a double bond.

“Substituted” groups are those in which one or more hydrogen atoms have been replaced with one or more non-hydrogen groups, provided that valence requirements are met and that a chemically stable compound results from the substitution.

“Alkyl” refers to straight chain and branched saturated hydrocarbon groups, generally having a specified number of carbon atoms (i.e., C₁₋₆ alkyl refers to an alkyl group having 1, 2, 3, 4, 5, or 6 carbon atoms). Examples of alkyl groups include, without limitation, methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkenyl” refers to straight chain and branched hydrocarbon groups having one or more unsaturated carbon-carbon bonds, and generally having a specified number of carbon atoms. Examples of alkenyl groups include, without limitation, ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include, without limitation, ethynyl, 1-propyn-1-yl, 2-propyn-1-yl, 1-butyn-1-yl, 3-butyn-1-yl, 3-butyn-2-yl, 2-butyn-1-yl, and the like.

“Alkanediyl” refers to divalent straight chain and branched aliphatic hydrocarbon groups, generally having a specified number of carbon atoms. Examples include, without limitation, methylene, 1,2-ethanediyl, 1,3-propanediyl, 1,4-butanediyl, 1,5-pentanediyl, 1,6-hexanediyl, and the like.

“Alkenediyl” refers to divalent, branched or unbranched, hydrocarbon groups having one or more unsaturated carbon-carbon bonds, and generally having a specified number of carbon atoms. Examples include, without limitation, ethene-1,2-diyl, propene-1,3-diyl, but-1-ene-1,4-diyl, but-2-ene-1,4-diyl, and the like.

“Alkynediyl” refers to divalent, branched or unbranched, hydrocarbon groups having one or more triple carbon-carbon bonds, and generally having a specified number of carbon atoms. Examples include, without limitation, ethyne-1,2-diyl, propyne-1,3-diyl, but-1-yne-1,4-diyl, but-2-yne-1,4-diyl, and the like.

“Alkyleneoxy,” “alkyleneamino,” and “alkylenethio” refer, respectively, to -alkyl-O—, -alkyl-NH—, and -alkyl-S—. Examples include, without limitation, methylenoxy, ethyleneoxy, 1,3-propyleneoxy, methyleneamino, ethyleneamino, 1,3-propyleneamino, methylenethio, ethylenethio, 1,3-propylenethio, and the like.

“Alkanoyl” refers to alkyl-C(O)—, where alkyl is defined above, and generally includes a specified number of carbon atoms, including the carbonyl carbon. Examples of alkanoyl groups include, without limitation, formyl, acetyl, propionyl, butyryl, pentanoyl, hexanoyl, and the like.

“Cycloalkyl” refers to saturated monocyclic and bicyclic hydrocarbon rings, generally having a specified number of carbon atoms that comprise the ring (i.e., C₃₋₇ cycloalkyl refers to a cycloalkyl group having 3, 4, 5, 6 or 7 carbon atoms as ring members). The cycloalkyl may be attached to a parent group or to a substrate at any ring atom, unless such attachment would violate valence requirements. Likewise, the cycloalkyl groups may include one or more non-hydrogen substituents unless such substitution would violate valence requirements. Useful substituents include, without limitation, alkyl, alkoxy, alkoxycarbonyl, and alkanoyl, as defined above, and hydroxy, mercapto, nitro, halogen, and amino.

Examples of monocyclic cycloalkyl groups include, without limitation, cyclopropyl, cyclobutyl, cyclopentyl, cyclohexyl, and the like. Examples of bicyclic cycloalkyl groups include, without limitation, bicyclo[1.1.0]butyl, bicyclo[1.1.1]pentyl, bicyclo[2.1.0]pentyl, bicyclo[2.1.1]hexyl, bicyclo[3.1.0]hexyl, bicyclo[2.2.1]heptyl, bicyclo[3.2.0]heptyl, bicyclo[3.1.1]heptyl, bicyclo[4.1.0]heptyl, bicyclo[2.2.2]octyl, bicyclo[3.2.1]octyl, bicyclo[4.1.1]octyl, bicyclo[3.3.0]octyl, bicyclo[4.2.0]octyl, bicyclo[3.3.1]nonyl, bicyclo[4.2.1]nonyl, bicyclo[4.3.0]nonyl, bicyclo[3.3.2]decyl, bicyclo[4.2.2]decyl, bicyclo[4.3.1]decyl, bicyclo[4.4.0]decyl, bicyclo[3.3.3]undecyl, bicyclo[4.3.2]undecyl, bicyclo[4.3.3]dodecyl, and the like.

“Cycloalkanoyl” refers to cycloalkyl-C(O)—, where cycloalkyl is defined above, and generally includes a specified number of carbon atoms, excluding the carbonyl carbon. Examples of cycloalkanoyl groups include, without limitation, cyclopropanoyl, cyclobutanoyl, cyclopentanoyl, cyclohexanoyl, cycloheptanoyl, and the like.

“Alkoxy,” “alkoxycarbonyl,” and “alkoxycarbonylalkyl” refer, respectively, to alkyl-O—, alkyl-O—C(O)—, and alkyl-O—C(O)-alkyl, where alkyl is defined above. Examples of alkoxy groups include, without limitation, methoxy, ethoxy, n-propoxy, i-propoxy, n-butoxy, s-butoxy, t-butoxy, n-pentoxy, s-pentoxy, and the like.

“Alkylaminocarbonyl,” “dialkylaminocarbonyl,” “alkylsulfonyl” “sulfonylaminoalkyl,” and “alkylsulfonylaminocarbonyl” refer, respectively, to alkyl-NH—C(O)—, alkyl₂-N—C(O)—, alkyl-S(O₂)—, HS(O₂)—NH-alkyl-, and alkyl-S(O)—NH—C(O)—, where alkyl is defined above.

“Halo,” “halogen” and “halogeno” may be used interchangeably, and refer to fluoro, chloro, bromo, and iodo.

“Haloalkyl” and “haloalkanoyl” refer, respectively, to alkyl or alkanoyl groups substituted with one or more halogen atoms, where alkyl and alkanoyl are defined above. Examples of haloalkyl and haloalkanoyl groups include, without limitation, trifluoromethyl, trichloromethyl, pentafluoroethyl, pentachloroethyl, trifluoroacetyl, trichloroacetyl, pentafluoropropionyl, pentachloropropionyl, and the like.

“Hydroxyalkyl” and “oxoalkyl” refer, respectively, to HO-alkyl and O=alkyl, where alkyl is defined above. Examples of hydroxyalkyl and oxoalkyl groups, include, without limitation, hydroxymethyl, hydroxyethyl, 3-hydroxypropyl, oxomethyl, oxoethyl, 3-oxopropyl, and the like.

“Aryl” and “arylene” refer to monovalent and divalent aromatic groups, respectively. Examples of aryl groups include, without limitation, phenyl, naphthyl, biphenyl, pyrenyl, anthracenyl, fluorenyl, and the like, which may be unsubstituted or substituted with 1 to 4 substituents such as alkyl, alkoxy, alkoxycarbonyl, alkanoyl, and cycloalkanoyl, as defined above, and hydroxy, mercapto, nitro, halogen, and amino.

“Arylalkyl” refers to aryl-alkyl, where aryl and alkyl are defined above. Examples include, without limitation, benzyl, fluorenylmethyl, and the like.

“Heterocycle” and “heterocyclyl” refer to saturated, partially unsaturated, or unsaturated monocyclic or bicyclic rings having from 5 to 7 or from 7 to 11 ring members, respectively. These groups have ring members made up of carbon atoms and from 1 to 4 heteroatoms that are independently nitrogen, oxygen or sulfur, and may include any bicyclic group in which any of the above-defined monocyclic heterocycles are fused to a benzene ring. The nitrogen and sulfur heteroatoms may optionally be oxidized. The heterocyclic ring may be attached to a parent group or to a substrate at any heteroatom or carbon atom unless such attachment would violate valence requirements. Likewise, any of the carbon or nitrogen ring members may include a non-hydrogen substituent unless such substitution would violate valence requirements. Useful substituents include, without limitation, alkyl, alkoxy, alkoxycarbonyl, alkanoyl, and cycloalkanoyl, as defined above, and hydroxy, mercapto, nitro, halogen, and amino.

Examples of heterocycles include, without limitation, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“Heteroaryl” and “heteroarylene” refer, respectively, to monovalent and divalent heterocycles or heterocyclyl groups, as defined above, which are aromatic. Heteroaryl and heteroarylene groups represent a subset of aryl and arylene groups, respectively.

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts), sulfonates (including tosylates, brosylates, nosylates, and mesylates), triflates, nonaflates, tresylates, halide ions, carboxylate anions, phenolate ions, and alkoxides. Some stronger bases, such as NH₂⁻ and OH⁻ can be made better leaving groups by treatment with an acid. Common electrofugal leaving groups include the proton, CO₂, and metals.

“Electron withdrawing group” refers to a substituent that pulls electron density from a neighboring atom or group of atoms via, for example, polarization or conjugation, and includes, for example, —C(O)R, —SO₂R, and —P(O)RR, where R and R′ are independently alkyl, aryl, or alkoxy. Useful electron withdrawing groups include, without limitation, cyano, alkanoyl, carboxy, alkoxycarbonyl, carbamoyl, alkylsulfonyl, and the like.

“Pharmaceutically acceptable salts, esters, amides, and prodrugs” refer to acid or base addition salts, esters, amides, zwitterionic forms, where possible, and prodrugs of claimed and disclosed compounds, which are within the scope of sound medical judgment, suitable for use in contact with the tissues of patients without undue toxicity, irritation, allergic response, and the like, commensurate with a reasonable benefit/risk ratio, and effective for their intended use.

Examples of pharmaceutically acceptable, non-toxic esters include, without limitation, C₁₋₆ alkyl esters, C₅₋₇ cycloalkyl esters, and arylalkyl esters of claimed and disclosed compounds, where alkyl, cycloalkyl, and aryl are defined above. Such esters may be prepared by conventional methods, as described, for example, in M. B. Smith and J. March, March's Advanced Organic Chemistry (5th Ed. 2001).

Examples of pharmaceutically acceptable, non-toxic amides include, without limitation, those derived from ammonia, primary C₁₋₆ alkyl amines, and secondary C₁₋₆ dialkyl or heterocyclyl amines of claimed and disclosed compounds, where alkyl and heterocyclyl are defined above. Such amides may be prepared by conventional methods, as described, for example, in March's Advanced Organic Chemistry.

“Prodrugs” refer to compounds having little or no pharmacological activity that can, when metabolized in vivo, undergo conversion to claimed or disclosed compounds having desired activity. For a discussion of prodrugs, see T. Higuchi and V. Stella, “Pro-drugs as Novel Delivery Systems,” ACS Symposium Series 14 (1975), E. B. Roche (ed.), Bioreversible Carriers in Drug Design (1987), and H. Bundgaar, Design of Prodrugs (1985). Prodrugs may be produced by replacing (formally) appropriate moieties present in the compounds of Formula 1 or Formula 10 with certain functional groups known as “pro-moieties” as described in H. Bundgaar. For example, for compounds containing a carboxylic acid group, a primary or secondary amine, or a hydroxyl group, pro-moieties would include an ester, an amide, or an ether group, respectively.

Table 1 lists abbreviations used throughout the specification.

TABLE 1
List of Abbreviations

| Abbreviation | Description |
|---|---|
| Ac | acetyl |
| ACN | acetonitrile |
| Aq | aqueous |
| p-BBB | para-bromobenzylbromide |
| 9-BBN | 9-(5-methyl-2-phenyl-oxazol-4-yl-propyl)-9-bora- |
| | bicyclo[3.3.1]nonane |
| Bn | benzyl |
| BnBr | benzylbromide |
| BrBn | bromobenzyl |
| BrAcOEt | ethyl bromoacetate |
| Bu | butyl |
| Bu₄NBr | tetrabutylammonium bromide |
| t-BuOK | potassium tertiary butyl oxide |
| t-BuOMe | tertiary butyl methyl ether |
| t-BuONa | Sodium tertiary butyl oxide |
| DBU | 1,8-diazabicyclo[5.4.0]undec-7-ene |
| DEAD | diethylazodicarboxylate |
| DIPEA | diisopropylethylamine |
| DMAP | 4-dimethylaminopyridine |
| DMF | dimethylformamide |
| DMSO | dimethylsulfoxide |
| e.e. | enantiomeric excess |
| Et | ethyl |
| ET₃N | triethylamine |
| EtOH | ethyl alcohol |
| EtOAc | ethyl acetate |
| h | hour |
| IAcOEt | ethyl iodoacetate |
| ID | internal diameter |
| LiHMDS | lithium hexamethyldisilazide |
| LTMP | lithium tetramethylpiperidide |
| LDA | lithium diisopropylamide |
| Me | methyl |
| MeI | methyl iodide |
| MeONa | sodium methoxide |
| min | minute |
| NMP | N-methylpyrrolidone |
| p-NO₂BnBr | p-nitrobenzylbromide |
| OD | outer diameter |
| PdCl₂(dppf)₂ | dichloro[1,1′- |
| | bis(diphenylphosphino)ferrocene]palladium (II) |
| | dichloromethane adduct |
| Ph | phenyl |
| Ph₃P | triphenylphosphine |
| PPAR | peroxisome proliferator-activated receptor |
| PTFE | polytetrafluoroethylene |
| RT | room temperature (approximately 20° C.-25° C.) |
| TFA | trifluoroacetic acid |
| THF | tetrahydrofuran |
| TLC | thin-layer chromatography |
| TRITON B | benzyltrimethylammonium hydroxide |

The present invention provides materials and methods for preparing compounds represented by Formula 1,

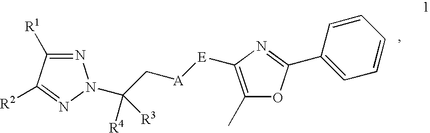

or by Formula 10,

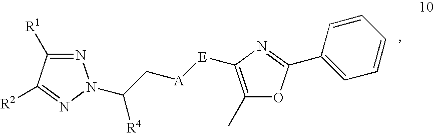

including pharmaceutically acceptable salts, esters, amides, and prodrugs thereof, in which R¹ and R² are independently hydrogen, halogen, aryl, benzoyl, C₁₋₆ alkyl, C₁₋₆ haloalkyl, C₁₋₆ alkanoyl, C₁₋₆ haloalkanoyl, or C₃₋₇ cycloalkanoyl;

R³ and R⁴ are electron-withdrawing groups, which may be the same or different;

E is C₁₋₆ alkyleneoxy, C₁₋₆ alkyleneamino, C₁₋₆ alkylenethio, C₁₋₆ alkanediyl, C₁₋₆ alkenediyl, or C₁₋₆ alkyndiyl; and

A is arylene or heteroarylene, each of which may have one or more non-hydrogen substituents, provided that when A is a five-member heteroarylene group, A is not linked to E through a heteroatom.

Particularly useful compounds represented by Formula 1 and Formula 10 include those in which R¹ and R² are each hydrogen, or those in which R³ and R⁴ are independently cyano, C₁₋₆ alkanoyl, carboxy, C₁₋₆ alkoxycarbonyl, carbamoyl, C₁₋₆ alkylaminocarbonyl, C₁₋₆ dialkylaminocarbonyl, sulfonylaminocarbonyl, C₁₋₆ alkylsulfonylaminocarbonyl, N-C₁₋₆ alkylsulfonyl-N-C₁₋₆ alkylaminocarbonyl, or C₁₋₆ alkylsulfonyl. Other useful compounds represented by Formula 1 and Formula 10 include those in which A is phenylene, especially p-phenylene, and E is methyleneoxy, ethyleneoxy, 1,3-propanediyl, 1,3-propenediyl, or 1,3-propynediyl.

Still other useful compounds represented by Formula 1 and Formula 10 include those in which R¹ and R² are each hydrogen, R³ and R⁴ are each C₁₋₆ alkoxycarbonyl, A is phenylene, and E is 1,3-propanediyl. As discussed above, an especially useful compound represented by Formula 10 is 3-{4-[3-(5-methyl-2-phenyl-oxazol-4-yl)-propyl]-phenyl}-2-[1,2,3]triazol-2-yl-propionic acid, which along with structurally-related compounds are known mixed PPAR α/γ agonists and potentially potent agents for treating non-insulin dependent diabetes.

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, materials and methods for installing and removing protecting groups, and a compilation of useful protecting groups for common functional groups, including amines, carboxylic acids, alcohols, ketones, aldehydes, and the like, see T. W. Greene and P. G. Wuts, Protecting Groups in Organic Chemistry (1999) and P. Kocienski, Protective Groups (2000), which are herein incorporated by reference in their entirety for all purposes.

In addition, some of the schemes and examples below may omit details of common reactions, including oxidations, reductions, and so on, which are known to persons of ordinary skill in the art of organic chemistry. The details of such reactions can be found in a number of treatises, including Richard Larock, Comprehensive Organic Transformations (1999), and the multi-volume series edited by Michael B. Smith and others, Compendium of Organic Synthetic Methods (1974-2003). Generally, starting materials and reagents may be obtained from commercial sources.

Scheme I illustrates a method of preparing compounds of Formula 1 and Formula 10. The method includes reacting a [1,2,3]triazole salt (Formula 2) with a first alkylating agent (Formula 3) in the presence of a solvent to yield a mixture of N-alkylated triazoles (Formula 4, 5). The triazole salt includes substituents R¹ and R², which are as defined above for the compound of Formula 1. More generally, and unless stated otherwise, when a particular substituent identifier (R¹, R², R³, etc.) is defined for the first time in connection with a formula, the same substituent identifier used in a subsequent formula will have the same meaning as in the earlier formula.

The triazole salt may be prepared separately or in situ (i.e., in the same vessel used to carry out the first alkylation) by contacting a [1,2,3]triazole having requisite substituents R¹ and R² with an appropriate base, such as NaH, t-BuONa, t-BuOK, and the like. For convenience, the triazole salt depicted in Scheme I shows a negative charge on the N2 atom, though in practice, the charge may be delocalized among the N1, N2, and N3 atoms. Similarly, Formula 2 depicts counter ion M with a 1+ charge, but M may be a 2+ ion. Useful M may thus include 1+ ions corresponding to Group 1 (alkali) metals (e.g., Na, K, Cs) or 2+ ions corresponding to Group 2 (alkaline earth) metals (e.g., Mg, Ca).

The first alkylating agent (Formula 3) includes substituents R³ and R⁴, which as described above in connection with the compound of Formula 1, are electron-withdrawing groups. The electron withdrawing groups are often the same, but may be different, and include, without limitation, cyano, C₁₋₆ alkanoyl, carboxy, C₁₋₆ alkoxycarbonyl, carbamoyl, C₁₋₆ alkylaminocarbonyl, C₁₋₆ dialkylaminocarbonyl, sulfonylaminocarbonyl, C₁₋₆ alkylsulfonylaminocarbonyl, N-C₁₋₆ alkylsulfonyl-N-C₁₋₆ alkylaminocarbonyl, or C₁₋₆ alkylsulfonyl. Particularly useful R³ and R⁴ include cyano, C₁₋₆ alkanoyl, and carboxy.

Surprisingly, the reaction of a [1,2,3]triazole salt (Formula 2) with a first alkylating agent (Formula 3) having a pair of electron withdrawing groups (R³ and R⁴) results in a product mixture having as major component an N2-alkylated triazole (Formula 4) in which the ratio of N2 alkylated triazole to N1-alkylated triazole (or triazoles) is greater than about 1:1. Moreover, and as shown below, this reaction methodology typically results in ratios of N2-alkylated triazole to N1-alkylated triazole of about 2:1 or greater, and in some cases, results in ratios of N2-alkylated triazole to N1-alkylated triazole of about 3:1 or greater.

This result is unexpected since it appears that there is no general and practical method for preparing N2-alkylated [1,2,3]triazoles. See, for example, K. T. Finley, 1,2,3: Triazole 1-17 (1981). Direct alkylation of unsubstituted [1,2,3]triazole with an alkyl halide and the like gives the N1-alkylated isomer as the major product. Apparently, the only reported exception is a Michael addition of an unsubstituted [1,2,3]triazole with a Michael acceptor, such as acrylonitrile, which is inappropriate for making compounds of Formula 1 and Formula 10. See Y. Tanaka and S. I. Miller, 29 Tetrahedron 3285 (1973) and H. Gold, 688 Liebigs Ann. 205 (1965). Furthermore, the scientific literature does not appear to disclose the preparation of N2-alkylated [1,2,3]triazoles through triazole ring formation.

The first alkylating agent provides other advantages. For example, the presence of two, though not necessarily the same, electron withdrawing groups, improves the yield of a subsequent alkylation described below. Additionally, since R³ and R⁴ of Formula 3 are non-hydrogen, the resulting molecular configuration prevents formation of bis-alkylated side-products of the subsequent alkylation, thereby further improving yield of the second alkylation. Particularly useful alkylating agents thus include β-dicarbonyl compounds, including dialkyl malonates (i.e., malonic acid dialkyl esters such as derivatives of dimethyl malonate and dimethyl malonate) or 3-oxo-C₄₋₉ alkanoic acid C₁₋₆ alkyl esters, including derivatives of ethyl acetoacetate.

In Formula 3, substituent X¹ is a leaving group that is displaced during the alkylation and can be halogen, sulfonate ester (including tosylates, brosylates, mesylates, and triflates), OP(O)(O-aryl)₂, etc. Particularly useful leaving groups include halogens such as chlorine and bromine. Thus, especially useful alkylating agents include dialkyl halomalonates, such as diethyl chloromalonate (i.e., 2-chloromalonic acid diethyl ester), dimethyl chloromalonate, diethyl bromomalonate, dimethyl bromomalonate, and the like.

Though the N2-alkylation of the triazole salt depends somewhat on choice of solvent and base, a variety of bases and polar organic solvents may be used. Useful solvents include acetone, EtOH, DMSO, THF, 1,4-dioxane, ACN, DMF, NMP, chloroform, chlorobenzene, and the like. Particularly useful solvents include polar aprotic solvents, such as DMF and ACN. When preparing the triazole salt of Formula 2 in situ, useful bases include various alkali and alkaline earth metal salts, such as NaH, t-BuONa, t-BuOK, and the like. Additionally or alternatively—i.e., when the triazole salt is prepared separately or is obtained from an external source—one may use other bases including Na₂CO₃, Et₃N, DBU, 4-dimethylaminopyridine (DMAP), diisopropylethylamine (DIPEA), benzyltrimethylammonium hydroxide (TRITON B), and similar non-nucleophilic (i.e., hindered) bases.

Although the N2-alkylation can be undertaken using substantially stoichiometric amounts of reactants, it is advantageous to carryout the reaction with an excess of the triazole salt (e.g., from about 1.1 equivalents to about 1.5 equivalents). The use of at least a slight excess of the triazole salt (e.g., about 1.1 equivalents) facilitates subsequent separation of products and reactants since the triazole salt can be stripped from the alkylation product mixture via aqueous extraction. More generally, and unless stated otherwise, the chemical transformations described throughout the specification can be carried out using substantially stoichiometric amounts of reactants or using an excess of one or more of the reactants. In addition, and unless stated otherwise, any reference in the disclosure to a stoichiometric range, a temperature range, a pH range, etc., includes the indicated endpoints.

As shown in some of the examples below, the temperature of the reaction mixture during and after admixing the first alkylating agent (Formula 3) and the triazole salt (Formula 2) may influence the ratio of N2-alkylated triazole to N1-alkylated triazole. Acceptable ratios of N2-alkylated triazole to N1-alkylated triazole ordinarily result for reaction temperatures between about −15° C. and 40° C. Depending on the particular reactants, higher yields of N2-alkylated triazole may result for reaction temperatures between about −15° C. and 20° C. Even higher yields of the N2-alkylated triazole may result for reaction temperatures between about −15° C. and 0° C. Since the reaction is exothermic, it is beneficial to add the alkylating agent to the reaction mixture through a series of partial additions, though extending the period of addition about ten-fold (e.g., from 30 min to 360 min) does not appear to significantly improve the ratio of N2-alkyated triazole.

As shown in Scheme I, the method also includes optionally reacting the mixture of N-alkylated triazoles (Formula 4 and Formula 5) with a second alkylating agent followed by contacting with a solvent, which as discussed below in connection with Scheme II, increases the fraction of the N2-alkylated triazole. Components of the reaction mixture are subsequently reacted with a third alkylating agent (Formula 7) in the presence of a base and solvent, to yield a compound of Formula 8. The third alkylating agent includes a linking group, A, which is as defined above for the compound of Formula 1, and a leaving group, X², which includes substituents defined above for X¹ of Formula 3. Particularly useful X² includes halogens such as chlorine and bromine. The third alkylating agent also includes a substituent, X³, which depending on a subsequent coupling reaction described below, may be a leaving group like X² or a nucleophilic group, such as hydroxy, amino, or thio.

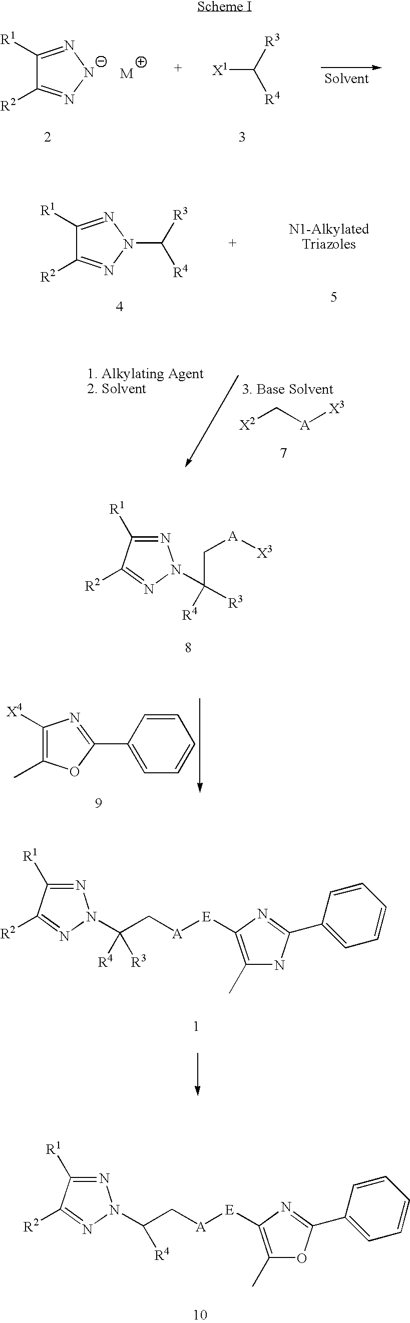

For the N2-alkylated triazole (Formula 4), the two electron withdrawing groups, R³ and R⁴, make a lone hydrogen atom that is bonded to a common carbon atom more acidic. This permits efficient alkylation under mild conditions using a relatively weak base (i.e., alkoxide or weaker base). For example, the N2-alkylated triazole of Formula 4 can be alkylated with p-bromobenzylbromide (p-BBB) at RT (room temperature) in an aprotic solvent such as DMF, THF, and the like, using K₂CO₃ as the base and a catalytic amount of Bu₄NBr. Harsher conditions and stronger bases can be used. For example, the N2-alkylated triazole of Formula 4 can also be alkylated with p-BBB in THF under reflux conditions, and using LiHMDS or other non-nucleophilic base, such as LTMP or LDA. Such conditions, however, are usually unnecessary.

As shown in Scheme I, following the third alkylation, the method includes coupling a compound of Formula 9 and the compound of Formula 8 to yield the compound of Formula 1. The compound of Formula 9, which may be prepared in accordance with methods disclosed in the '553 Application, includes substituent X⁴, which depending on the nature of the coupling reaction may be a C₁₋₆ hydroxyalkyl, C₁₋₆ oxoalkyl, C₁₋₆ haloalkyl, C₂₋₆ alkenyl, and C₂₋₆ alkynyl. For example, when X³ is hydroxy and X⁴ is C₁₋₆ hydroxyalkyl (e.g., hydroxyethyl), the compounds of Formula 8 and 9 can be coupled under Mitsunobu conditions (DEAD, Ph₃P, THF) to yield the compound of Formula 1 in which E is C₁₋₆ alkyleneoxy (e.g., ethyleneoxy). When X³ is hydroxy or thio, and X⁴ is C₁₋₆ haloalkyl (e.g., bromoethyl) the compounds of Formula 8 and 9 can be coupled in the presence of a base (e.g., MeONa) to yield the compound of Formula 1 in which E is C₁₋₆ alkyleneoxy (e.g., ethyleneoxy) or C₁₋₆ alkylenethio (e.g., ethylenethio), respectively. Additionally, when X³ is amino and X⁴ is C₁₋₆ oxoalkyl (e.g., oxoethyl), the compounds of Formula 8 and 9 can be reacted in the presence of catalytic amounts of an acid to form an imine intermediate, which is subsequently reduced to yield the compound of Formula 1 in which E is C₁₋₆ alkyleneamino (e.g., ethyleneamino).

The compounds of Formula 8 and 9 can be coupled in other ways. For example, when X³ is a leaving group (e.g., triflate) and X⁴ is C₂₋₆ alkenyl (e.g., prop-1-ene-3-yl) or C₂₋₆ alkynyl (e.g., prop-1-yne-3-yl) the compounds of Formula 8 and 9 can be coupled in the presence of an organometallic catalyst to yield the compound of Formula 1 in which E is C₁₋₆ alkenediyl (e.g., propenediyl) or C₁₋₆ alkyndiyl (propynediyl). Alternatively, when X⁴ is C₂₋₆ alkenyl or C₂₋₆ alkynyl, the compound of Formula 9 can be reacted with a hydroboration agent, such as 9-BBN, to yield an alkyl- or alkenyl-9-BBN adduct, which is subsequently combined with the compound of Formula 8 (X³ is halogen or triflate) to yield the compound of Formula 1 in which E is C₁₋₆ alkanediyl or C₁₋₆ alkenediyl. The hydroboration is carried out at RT in a polar aprotic solvent, such as THF, and the Suzuki coupling is carried out at RT in a mixed solvent, DMF-H₂O, and in the presence of a base, CsCO₃, and a catalyst, PdCl₂(dppf), Ph₃As. For descriptions of other useful couplings, see the '553 Application.

Following the coupling of the compounds of Formula 8 and 9, the method optionally provides for removal or transformation of R³ or R⁴ in Formula 1 (e.g., replacement with a hydrogen atom). For instance, when R³ and R⁴ are both alkoxycarbonyl—as would be the case when the first alkylating agent (Formula 3) is a malonate derivative—R³ (or R⁴) can be removed by hydrolysis of the ester moieties, followed by decarboxylation to yield the compound of Formula 10, where R⁴ (or R³) is CO₂. When R³ (or R⁴) is an alkanoyl and R⁴ (or R³) is an alkoxycarbonyl—as would be the case when the first alkylating agent is an acetoacetate derivative—the unwanted alkanoyl group can be removed by either base or acid hydrolysis. Similarly, when R³ and R⁴ are both cyano groups, they can be hydrolyzed (in acid or base) to give a carboxylic diacid, which is followed by decarboxylation to give compound of Formula 10.

Scheme II provides further details of the second alkylation. As described above, the method optionally includes reacting the mixture of N-alkylated triazoles of Formula 4 and Formula 5 with a second alkylating agent (Formula 11), which unexpectedly and preferentially converts the N1-alkylated triazole (or triazoles) of Formula 5 to one or more N1,N3-bisalkylated triazolium intermediates (Formula 12). The resulting reaction mixture, which includes the N1,N3-bisalkylated triazolium intermediate and the N2-alkylated triazole, is subsequently contacted with an appropriate solvent. Because of the zwitterionic nature of the N1,N3-bisalkylated triazolium intermediate, contacting the reaction mixture with a less polar solvent, including esters (e.g., EtOAc), ethers (e.g., t-BuOMe), aromatic solvents (e.g., toluene, benzene), and the like, causes the N1,N3-bisalkylated triazolium intermediate to precipitate out of solution while leaving the desired N2-alkylated triazole in solution. Filtering the reaction mixture removes the N1,N3-bisalkylated triazolium precipitate, and results in a substantial increase in the fraction of the N2-alkylated triazole in the reaction mixture (filtrate).

As shown in the Examples below, a wide variety of alkylating agents can be used to convert the N1-alkylated triazoles of Formula 5 to the N1,N3-bisalkylated triazolium intermediates of Formula 12. In the expression for the second alkylating agent (Formula 11) useful R⁵ include, but are not limited to substituted or unsubstituted C₁₋₆ alkyl, C₁₋₆ alkoxycarbonyl, C₁₋₆ alkoxycarbonylalkyl, and arylalkyl. Particularly useful R⁵ include Me, EtOAc, Bn, BrBn, and NO₂Bn. In Formula 11, X⁵ is a leaving group that is displaced during alkylation and includes groups defined above for X¹ of Formula 3, including bromine and iodine. Exemplary second alkylating agents thus include, without limitation, methyl iodide, ethyl bromoacetate, ethyl iodoacetate, benzylbromide, p-nitrobenzylbromide, and p-BBB.

The second alkylation can be run in one or more solvents (e.g., THF, DMF, etc.) and in the presence of one or more bases (e.g., KHCO₃), which may be the same as those described above for the first alkylation. As shown in the Examples, however, in some cases carrying out the second alkylation without solvent or base (neat) may improve the conversion of the N1-alkylated triazoles of Formula 5 to the N1,N3-bisalkylated triazolium intermediates of Formula 12. This surprising result leads to higher fractions of the N2-alkylated triazole in the reaction mixture. For instance, alkylation of certain N1-alkylated triazoles (R¹, R² are each H and R³, R⁴ are each ethoxycarbonyl in Formula 5) with MeI or p-BBB in the presence of solvent (THF or DMF) or solvent and base (KHCO₃), results in an increase in the molar ratio of N2- to N1-alkylated triazoles from 1.5/1 to between about 1.6/1 and 7/1, whereas alkylation in the absence of solvent or base results in an increase in the molar ratio from 1.5/1 to between about 4.8/1 and 10/1.

As shown in Scheme III, the order of the second and third alkylations can be reversed. For example, the method may alternatively include reacting the N-alkylated triazoles of Formula 4 and Formula 5 with the alkylating agent of Formula 7 in the presence of a base and solvent, to yield, in addition to the N2-alkylated triazole of Formula 8 discussed above, one or more N1-alkylated triazoles (Formula 14). The N1-alkylated triazoles of Formula 14 are subsequently reacted with the alkylating agent of Formula 11 to yield N1,N3-bisalkylated triazolium intermediates (Formula 16). The resulting reaction mixture is subsequently contacted with an appropriate solvent, which causes the N1,N3-bisalkylated triazolium intermediates of Formula 16 to precipitate out of solution while leaving the desired N2-alkylated triazole of Formula 8 in solution. Reagents and conditions used in the second and third alkylations shown in Scheme I and in Scheme II can also be used in the corresponding alkylations depicted in Scheme III.

Filtering the reaction mixture removes the N1,N3-bisalkylated triazolium precipitate, and results in a mixture having a substantial excess of the N2-alkylated triazole of Formula 8 relative to the N1-alkylated triazole of Formula 14. For instance, treatment of a 1.5/1 molar mixture of N2- and N1-alkylated triazoles (R¹, R² are each H and R³, R⁴ are each ethoxycarbonyl in Formula 4 and Formula 5) with p-BBB in the presence of K₂CO₃ in DMF at RT gives a mixture (98% yield) of N2- and N1-alkylated triazoles (Formula 8 and Formula 14 with A and X³ being Bn and Br, respectively). Treatment of the resulting reaction mixture with BnBr at a temperature between about 60° C. and 70° C. for 24 h and subsequently contact with t-BuOMe precipitates the undesirable bis-alkylated triazolium derivatives (Formula 16). Filtering out the bis-alkylated triazolium derivatives gives a 10/1 molar mixture of N2- and N1-alkylated triazoles of Formula 8 and of Formula 14, respectively.

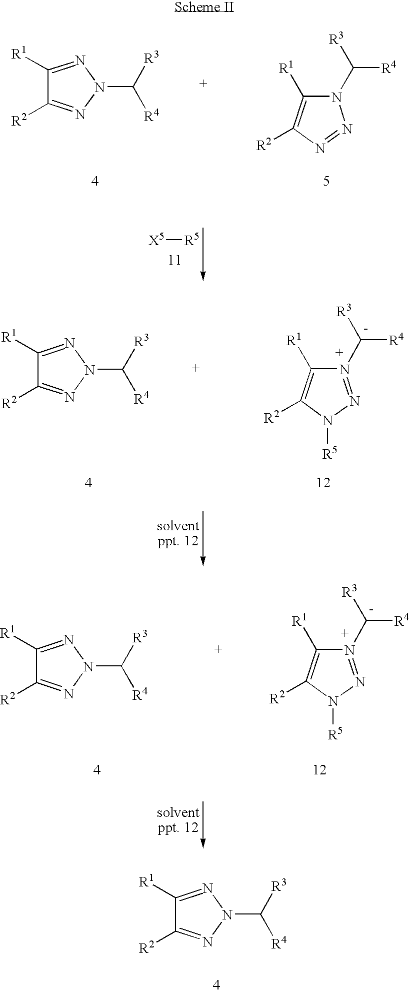

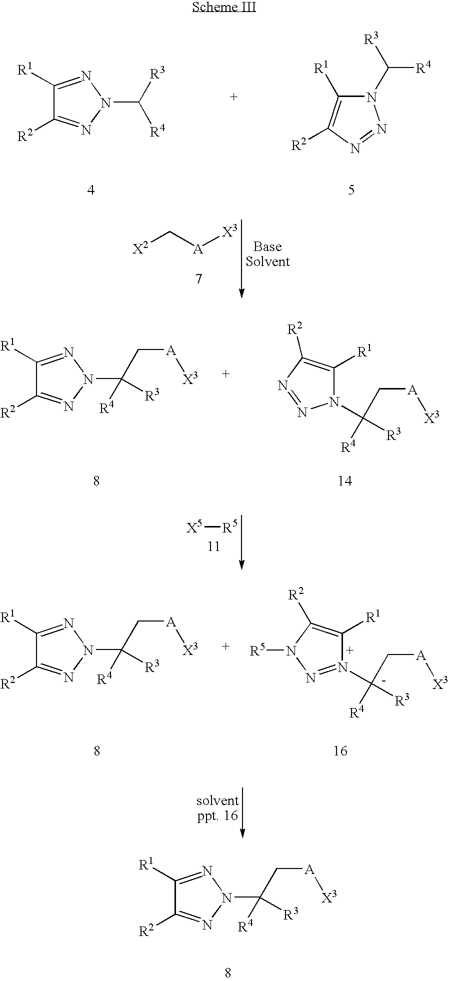

Many of the compounds described in this disclosure, including those represented by Formula 1 and Formula 10, are capable of forming pharmaceutically acceptable salts. These salts include, without limitation, acid addition salts (including diacids) and base salts. Pharmaceutically acceptable acid addition salts may include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts may thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like.

Pharmaceutically acceptable base salts may include nontoxic salts derived from bases, including metal cations, such as an alkali or alkaline earth metal cation, as well as amines. Examples of suitable metal cations include, without limitation, sodium cations (Na⁺), potassium cations (K⁺), magnesium cations (Mg²⁺), calcium cations (Ca²⁺), and the like. Examples of suitable amines include, without limitation, N,N′-dibenzylethylenediamine, chloroprocaine, choline, diethanolamine, dicyclohexylamine, ethylenediamine, N-methylglucamine, and procaine. For a discussion of useful acid addition and base salts, see S. M. Berge et al., “Pharmaceutical Salts,” 66 J. of Pharm. Sci., 1-19 (1977); see also Stahl and Wermuth, Handbook of Pharmaceutical Salts: Properties, Selection, and Use (2002).

One may prepare a pharmaceutically acceptable acid addition salt (or base salt) by contacting a compound's free base (or free acid) with a sufficient amount of a desired acid (or base) to produce a nontoxic salt. One may then isolate the salt by filtration if it precipitates from solution, or by evaporation to recover the salt. One may also regenerate the free base (or free acid) by contacting the acid addition salt with a base (or the base salt with an acid). Though certain physical properties of the free base (or free acid) and its respective acid addition salt (or base salt) may differ (e.g., solubility, crystal structure, hygroscopicity, etc.), a compound's free base and acid addition salt (or its free acid and base salt) are otherwise equivalent for purposes of this disclosure. The degree of ionization in the resulting salt may vary from completely ionized to almost non-ionized

Additionally, certain compounds of this disclosure, including those represented by Formula 1 and Formula 10, may exist as an unsolvated form or as a solvated form, including hydrated forms. Pharmaceutically acceptable solvates include hydrates and solvates in which the crystallization solvent may be isotopically substituted, e.g. D₂O, d₆-acetone, d₆-DMSO, etc. Generally, the solvated forms, including hydrated forms, are equivalent to unsolvated forms for the purposes of this disclosure. Thus, unless expressly noted, all references to the free base, the free acid or the unsolvated form of a compound also includes the corresponding acid addition salt, base salt or solvated form of the compound.

Some of the compounds disclosed in this specification may also contain one or more asymmetric carbon atoms and therefore may exist as optically active stereoisomers (i.e., pairs of enantiomers). Some of the compounds may also contain an alkenyl or cyclic group, so that cis/trans (or Z/E) stereoisomers (i.e., pairs of diastereoisomers) are possible. Still other compounds may exist as one or more pairs of diastereoisomers in which each diastereoisomer exists as one or more pairs of enantiomers. Finally, some of the compounds may contain a keto or oxime group, so that tautomerism may occur. In such cases, the scope of the present invention includes individual stereoisomers of the disclosed compound, as well as its tautomeric forms (if appropriate).

Individual enantiomers may be prepared or isolated by known techniques, such as conversion of an appropriate optically-pure precursor, resolution of the racemate (or the racemate of a salt or derivative) using, for example, chiral HPLC, or fractional crystallization of diastereoisomeric salts formed by reaction of the racemate with a suitable optically active acid or base (e.g., tartaric acid). Diastereoisomers may be separated by known techniques, such as fractional crystallization and chromatography.

For example, and as noted above, useful compounds of Formula 1 (and 10) include 3-{4-[3-(5-methyl-2-phenyl-oxazol-4-yl)-propyl]-phenyl}-2-[1 ,2,3]triazol-2-yl-propionic acid (Formula 29, Example 52), which has a stereogenic center and therefore comprises a pair of optically active stereoisomers. The S-enantiomer (Formula 30, Example 53) can be isolated by chiral HPLC separation using a CHIRALPAK AD column having a mobile phase of n-heptane, EtOH, and TFA (75/25/0.1). The column eluate can be neutralized with triethylamine, which yields the S-enantiomer as en Et₃N salt in good enantiomeric excess (95% e.e.). The major impurity is an Et₃N salt of TFA, which can be removed via extraction with ethyl acetate and water at pH 4. Recrystallization from acetonitrile improves the optical purity of the S-enantiomer to greater than 99% e.e.

The disclosed compounds also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as ³⁵S; isotopes of fluorine, such as ¹⁸F; and isotopes of chlorine, such as ³⁶Cl. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies.

### EXAMPLES

The following examples are intended to be illustrative and non-limiting, and represent specific embodiments of the present invention.

### Example 1

### Preparation of 2-[1,2,3]triazol-2-yl-malonic acid diethyl ester (Formula 20) and 2-[1,2,3]triazol-1-yl-malonic acid diethyl ester (Formula 21)

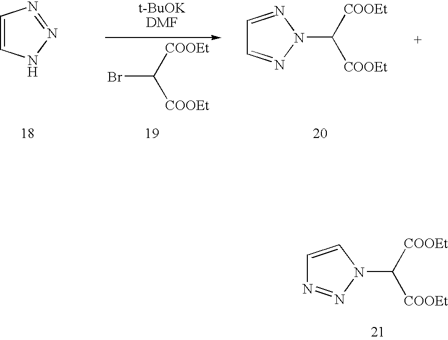

Sodium t-butyl oxide (123.5 g, 1.28 mole, ALDRICH) was added in 4 equal portions to a solution of [1,2,3]triazole (90.44 g, 1.3 mole, CHONTECH, Formula 18) and dry DMF (1 L, BAKERDRY) in a 2 L 3-neck flask, which was equipped with mechanical stirrer, thermometer, dropping funnel, nitrogen inlet, and ice bath. The reaction temperature rose to 17° C. during the addition. After the addition, the ice bath was removed and the reaction mixture was stirred at RT for 40 min to give a clear solution. The solution was cooled to about −10° C. with an ACN/dry ice bath. Diethyl bromomalonate (211.6 mL, 1.18 mole, Formula 19) was added to the sodium salt of [1,2,3]triazole over a period of 18 min while maintaining the temperature of the reaction mixture below 0° C. Following the addition, the dry ice bath was removed and the reaction mixture was stirred at RT for 24 h. The reaction mixture was poured into water (1 L) and extracted with t-BuOMe (3.5 L). The organic layer was washed with saturated NaHCO₃ (800 mL), saturated NaCl (2×500 mL), and dried over anhydrous MgSO₄. The solvent was removed to give a yellow oil (215 g). The aqueous layers were combined, extracted again with t-BuOMe (600 mL), and worked up the same way as above to give additional oil (16 g). The two crops were combined to give a mixture of the titled compounds (231 g, 86%). ¹H-NMR showed the ratio of the N2- to N1-alkylated isomers (compounds of Formula 20 and Formula 21, respectively) was 2.1/1.

### Examples 2-14

### Preparation of 2-[1,2,3]triazol-2-yl-malonic acid diethyl ester (Formula 20) and 2-[1,2,3]triazol-1-yl-malonic acid diethyl ester (Formula 21)

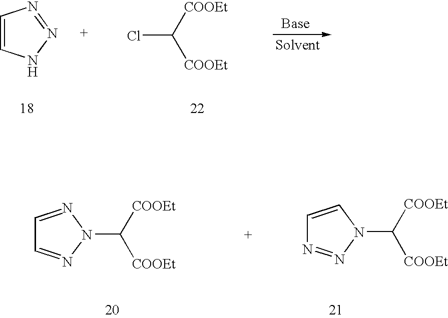

Table 2 lists conditions, reagents, and N2/N1 isomer product ratios for alkylations of [1,2,3]triazole (Formula 18) with diethyl chloromalonate (Formula 22). Using base and solvent pairs provided in Table 2, each of the reactions was carried out in a manner similar to that described in Example 1, though at smaller scale. Furthermore, only Example 12 included in situ preparation of the sodium salt of [1,2,3]triazole. Each of the reactions was run with a slight excess of [1,2,3]triazole relative to diethyl chloromalonate (i.e., about a 1.1/1.0 molar ratio). The title compounds were separated by HPLC and the areas of the resulting chromatograms were used to calculate the ratios of the N2- to N1-alkylation products (Formula 20 and Formula 21, respectively).

TABLE 2
Alkylation of [1,2,3]triazole with diethyl chloromalonate

| Example | Base | Solvent | Temperature | Time, h | N2/N1¹ |
|---|---|---|---|---|---|
| 2 | DIPEA | DMSO | RT | 16 | 1/5.4 |
| 3 | DIPEA | DMF | RT | 16 | 1/5.8 |
| 4 | DIPEA | ACN | RT | 16 | 1/6.7 |
| 5 | DIPEA | THF | RT | 16 | 1/6.7 |
| 6 | DIPEA | Dioxane | RT | 16 | 1/7.7 |
| 7 | TRITON B | DMSO | RT | 24 | 1/3.8 |
| 8 | TRITON B | DMF | RT | 24 | 1/3.6 |
| 9 | TRITON B | ACN | RT | 24 | 1/2.6 |
| 10 | TRITON B | THF | RT | 24 | 1/2.8 |
| 11 | TRITON B | Dioxane | RT | 24 | 1/2.5 |
| 12 | t-BuONa² | ACN | RT | 16 | 1/0.89 |
| 13 | Na salt³ | DMSO | RT | 16 | 1/1.3 |
| 14 | Na salt³ | DMF | RT | 16 | 1/0.81 |

¹Ratio of HPLC chromatogram areas
²Sodium salt of [1,2,3]triazole prepared in situ like Example 1
³Sodium salt of [1,2,3]triazole prepared separately

### Examples 15-25

### Preparation of 2-[1,2,3]triazol-2-yl-malonic acid diethyl ester (Formula 20) and 2-[1,2,3]triazol-1-yl-malonic acid diethyl ester (Formula 21)

Table 3 lists conditions, reagents, and N2/N1 isomer product ratios for alkylations of [1,2,3]triazole (including sodium, potassium, and lithium salts) with diethyl bromomalonate (Formula 19). Using base and solvent pairs provided in Table 3, each of the reactions was carried out in a manner similar to that described in Example 1, though at smaller scale. Furthermore, only Examples 21, 22, and 24 included in-situ preparation of the sodium or potassium salt of [1,2,3]triazole. Each of the reactions was run with a slight excess of [1,2,3]triazole relative to diethyl bromomalonate (i.e., about a 1.1/1 molar ratio). The title compounds were separated by HPLC and the areas of the resulting chromatograms were used to calculate the ratios of the N2- to N1-alkylation products (Formula 20 and Formula 21, respectively).

TABLE 3
Alkylation of [1,2,3]triazole with diethyl bromomalonate

| Example | Base | Solvent | Temperature | Time, h | N2/N1¹ |
|---|---|---|---|---|---|
| 15 | Na salt² | DMSO | RT | 16 | 1/1.7 |
| 16 | Na salt² | DMF | RT | 16 | 1/0.76 |
| 17 | Na salt² | THF | RT | 23 | 1/1.1 |
| 18 | Na salt² | ACN | RT | 24 | 1/0.76 |
| 19 | Na salt² | CHCl₃ | RT | 24 | 1/3.7 |
| 20 | Na salt² | Chlorobenzene | RT | 24 | 1/2.8 |
| 21 | t-BuONa³ | EtOH | RT | 16 | 1/1.2 |
| 22 | t-BuONa³ | DMF | 0° C. to RT | 22 | 1/0.65 |
| 23 | Li salt⁴ | DMF | 0° C. to RT | 5 | 1/3.9 |
| 24 | t-BuOK³ | DMF | 0° C. to RT | 22 | 1/0.74 |
| 25 | No base | EtOH | Reflux | 16 | ≅0 |

¹Ratio of HPLC chromatogram areas
²Sodium salt of [1,2,3]triazole prepared separately
³Sodium salt of [1,2,3]triazole prepared in situ like Example 1 using indicated base
⁴Lithium salt of [1,2,3]triazole prepared separately

### Examples 26-31

### Preparation of 2-[1,2,3]triazol-2-yl-malonic acid diethyl ester (Formula 20) and 2-[1,2,3]triazol-1-yl-malonic acid diethyl ester (Formula 21)

Table 4 lists conditions (time and temperature during and after addition of the alkylation agent), reagents (bases), N2/N1 isomer product ratios, and crude product yields for alkylations of [1,2,3]triazole (Formula 18) with diethyl bromomalonate (Formula 19). Each of the reactions was carried out in DMF and in a manner similar to that described in Example 1. The reactions were run with a slight excess of [1,2,3]triazole relative to diethyl bromomalonate (i.e., about a 1.1/1 molar ratio). The ratios of the N2- to N1-alkylation products (Formula 20 and Formula 21, respectively) were obtained using proton NMR.

TABLE 4
Alkylation of [1,2,3]triazole with diethyl bromomalonate

| | | Time and Temperature² | | | |
|---|---|---|---|---|---|
| Example | Base¹ | During | Following | N2/N1³ | Yield⁴ |
| 26 | NaH | 30 min @ | 22 h @ | 1/0.7 | 143 g |
| | | 10° C. to 16° C. | RT | | 95% |
| 27 | NaH | 30 min @ | 22 h @ | 1/0.71 | 128 g |
| | | 40° C. | RT | | 95% |
| 28 | NaH | 50 min @ | 20 h @ −5° C. | 1/0.42 | 21 g |
| | | −15° C. to 7° C. | 22 h @ RT | | 89% |
| 29 | t-BuONa | 20 min @ | 24 h @ | 1/0.47 | 231 g |
| | | −12° C. to −0.8° C. | RT | | 86%) |
| 30 | t-BuONa | 70 min @ | 48 h @ | 1/0.45 | 732 g |
| | | −12° C. to −3° C. | RT | | 88% |
| 31 | t-BuONa | 360 min @ | 24 h @ 0° C. | 1/0.56 | 624 g |
| | | −7° C. to −3° C. | 48 h @ RT | | 88% |

¹Sodium salt of [1,2,3]triazole prepared in situ as in Example 1 using indicated base
²Time and temperature during and following addition of the compound of Formula 19
³By ¹H-NMR
⁴Yield of crude product mixture in mass and % of limiting reactant (Formula 19)

### Example 32

### Isolation of 2-[1,2,3]triazol-2-yl-malonic acid diethyl ester (Formula 20) from a mixture of 2-[1,2,3]triazol-2-yl-malonic acid diethyl ester and 2-[1,2,3]triazol-1-yl-malonic acid diethyl ester (Formula 21)

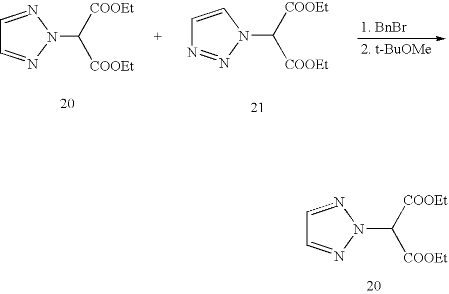

Benzyl bromide (57.4 g, 0.336 mole, ALDRICH) was added to a mixture of the compounds of Formula 20 and 21 (230 g, N2/N1=2.1/1, about 0.33 mole of the compound of Formula 21) and heated to a temperature of about 63° C. for 63 h using an oil bath. ¹H-NMR showed that the ratio of the compound of Formula 20 to the compound of Formula 21 (N2/N1) was 12/1. Additional BnBr (8 mL, 0.067 mole) was added and continuously heated at 63° C. for 50 h. ¹H-NMR showed that N2/N1 was 25/1. Additional BnBr (3 mL) was added and heated at 63° C. for 17 h. ¹H-NMR showed that N2/N1 was 41/1. Ethyl acetate (1.5 L) was added slowly to the reaction mixture to give an orange suspension with a gummy solid sticking to the sides of the flask. The suspension was filtered through a pad of CELITE and washed with EtOAc (100 mL). The filtrate was washed with saturated NaHCO₃ (2×600 mL), saturated NaCl, and dried over anhydrous MgSO₄. The solution was then filtered through a pad of silica gel (130 g silica gel 60, column: 9×3.5 cm, OD×H), and the silica gel cake was washed with ethyl acetate (50 mL). The solvent was removed to give brown oil, which was diluted with t-BuOMe (500 mL) to give a clear brown solution. The solution was again filtered through a pad of silica gel (90 g silica gel 60, column: 7.5×3 cm, OD×H), and the silica gel cake was washed t-BuOMe (100 mL). The solvent was removed to give a brown oil (177.3 g). ¹H-NMR showed little improvement of purity between the first and second silica gel filtration. The oil was heated in hexane (1 L) at reflux with stirring for 20 min. The bi-layer was cooled to RT overnight and seeded with a crystal of Formula 20, resulting in a solid bottom layer and a clear liquid top layer, which was decanted off and saved. The bottom solid cake was dispersed and heated in hexane (500 mL), cooled to RT, and seeded with a crystal of Formula 20, which again resulted in a bottom solid layer and a clear liquid top layer that was decanted off and saved. The solid cake was dried under vacuum to give 132.9 g of the desired N2-alkylated triazole (Formula 20). The decanted liquid was kept in a refrigerator overnight to give additional N2-alkylated triazole as white long needle crystals (5.1 g). The two crops were combined (total 60% yield), made homogenous by dissolving in dichloromethane followed by removal of solvent. ¹H-NMR showed the ratio of N2/N1 isomers was 60/1. The overall two-step yield from diethyl bromomalonate was 51.5%. ¹H-NMR (CDCl₃) δ 7.73 (s, 2H), 6.06 (s, 1H), 4.33 (q, J=7.3 Hz, 2H), 1.30 (t, J=7.4 Hz, 3H). MS (Scan AP+) 228 m/z (M+1, 100%). Calculated for C₉H₁₃N₃O₄: C 47.57, H 5.77, N 18.49; found: C, 47.54, H, 5.64, N, 18.21.

### Example 33-46

### Isolation of 2-[1,2,3]triazol-2-yl-malonic acid diethyl ester (Formula 20) from a mixture of 2-[1,2,3]triazol-2-yl-malonic acid diethyl ester and 2-[1,2,3]triazol-1-yl-malonic acid diethyl ester (Formula 21)

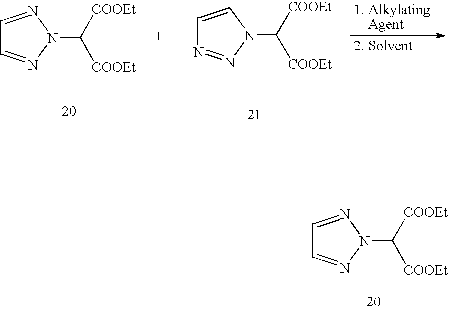

Table 5 lists alkylation agents, solvents, reaction time and temperature, and initial and final N2/N1 ratios for isolating 2-[1,2,3]triazol-2-yl-malonic acid diethyl ester from a mixture of 2-[1,2,3]triazol-2-yl-malonic acid diethyl ester and 2-[1,2,3]triazol-1-yl-malonic acid diethyl ester. Each of the alkylations and subsequent separations were carried out in a manner similar to the isolation methodology described in Example 32, though at different scale. The ratios of the N2- to N1-alkylation products (Formula 20 and Formula 21, respectively) were obtained using proton NMR.

TABLE 5
Isolation of 2-[1,2,3]triazol-2-yl-malonic acid diethyl ester

| | Alkylating | | Temperature | | Initial | Final |
|---|---|---|---|---|---|---|
| Example | Agent | Solvent | ° C. | Time h | N2/N1¹ | N2/N1¹ |
| 33 | MeI | THF | 60 | 36 | 1.5/1 | 1.8/1 |
| 34 | MeI | Neat | 70 | 6 | 1.5/1 | 4.8/1 |
| 35 | BrAcOEt | Neat | 70 | 6 | 1.5/1 | 2.3/1 |
| 36 | IAcOEt | Neat | 70 | 6 | 1.5/1 | 5.5/1 |
| 37 | p-BBB | DMF | 100 | 24 | 1.5/1 | 7.0/1 |
| 38 | p-BBB | DMF² | 60 | 36 | 1.5/1 | Messy |
| 39 | p-BBB | THF | 60 | 36 | 1.5/1 | 1.6/1 |
| 40 | p-BBB | THF² | 60 | 36 | 1.5/1 | 2.0/1 |
| 41 | p-BBB | Neat | 100 | 4 | 1.5/1 | 10.0/1 |
| 42 | p-NO₂BnBr | Neat | 70 | 6 | 1.5/1 | 6.2/1 |
| 43 | BnBr | Neat | 60 | 19 | 1.5/1 | 5.5/1 |
| 44 | BnBr | Neat | 70 | 5 | 1.5/1 | 7.8/1 |
| 45 | BnBr | Neat | 80 | 3 | 1.5/1 | 6.0/1 |
| 46 | BnBr | Neat | 70 | 19 | Only N2 | No Rxn |

¹By ¹H-NMR
²With KHCO₃

### Example 47

### Preparation of 2-(4-bromo-benzyl)-2-[1,2,3]triazol-2-yl-malonic acid diethyl ester (Formula 23) and 3-(bis-ethoxycarbonyl-methyl)-1-(4-bromo-benzyl)-3H-[1,2,3]triazol-1-ium (Formula 24)

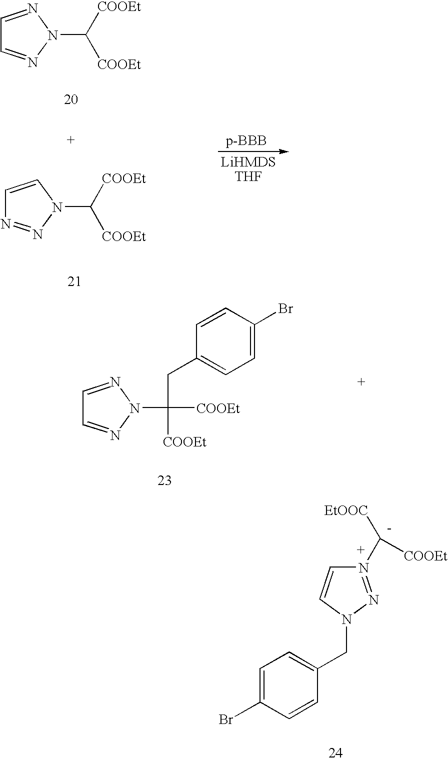

A solution of 2-[1,2,3]triazol-2-yl-malonic acid diethyl ester (Formula 20) and 2-[1,2,3]triazol-1-yl-malonic acid diethyl ester (Formula 21) (1/0.25, 11.1 g, 48.9 mmol) in dry THF (100 mL) was dried with 4 A molecular sieve (3 g) at RT for 2 h. The solution was then transferred to another dry flask through a cannula. The solution was cooled in an ice bath and LiHMDS (49.8 mL, 1 M, 49.8 mmol) in THF was added drop-wise under nitrogen. After the addition, the dark brown solution was moved to RT and stirred for 30 min. Then p-BBB (12.6 g, 50.3 mmol) was added in 1 portion. The reaction mixture was heated to reflux for 20 h. HPLC showed that the reaction was complete. After cooling to RT, the reaction mixture was diluted with EtOAc (500 mL) and washed with water (2×200 mL). Immediately after the addition of water, solids formed, which stayed mostly in the organic layer. The organic layer was washed with saturated NaCl, which resulted in more solids precipitating out of the organic layer. After separation of the two layers, the organic suspension was filtered to give a white solid (1.74 g). ¹H-NMR showed it was pure compound of Formula 24: mp=230° C. to 232° C.; ¹H-NMR (CDCl₃) δ 8.01 (s, 1H), 7.73 (s, 1H), 7.59 (d, J=8.6 Hz, 2H), 7.24 (d, J=9 Hz, 2H), 5.61 (s, 2H), 4.16 (q, J=7.1 Hz, 4H), 1.26 (t, J=7.1 Hz, 6H); MS (Scan AP+) 396 m/z (M+1, 100%). The filtrate was concentrated to give a brown paste. The paste was heated in a mixture of t-BuOMe/hexane (200 mL/70 mL) to reflux. After cooling to RT, the resulting suspension was filtered to give a brown solid (1.17 g). ¹H-NMR showed it was mostly compound of Formula 24. The total amount of the compound of Formula 24 isolated was about 15%. The filtrate was subsequently concentrated and purified by silica gel chromatography to give the compound of Formula 23 as a brown oil (12.1 g, 62%).

### Example 48

### Preparation of 2-(4-bromo-benzyl)-2-[1,2,3]triazol-2-yl-malonic acid diethyl ester (Formula 23) and 2-(4-bromo-benzyl)-2-[1,2,3]triazol-1-yl-malonic acid diethyl ester (Formula 25)

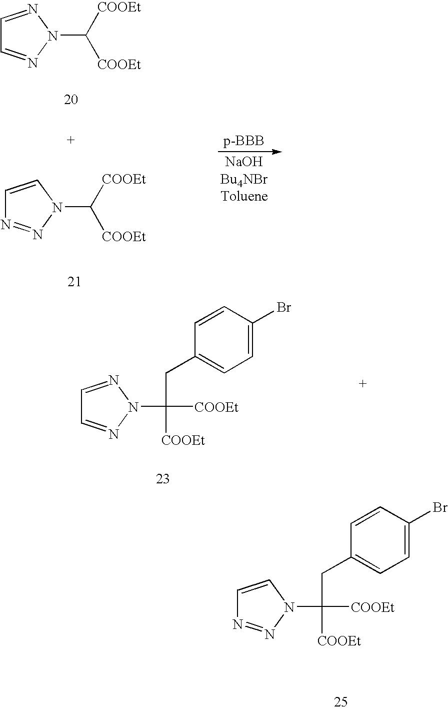

To a solution of 2-[1,2,3]triazol-2-yl-malonic acid diethyl ester (Formula 20) and 2-[1,2,3]triazol-1-yl-malonic acid diethyl ester (Formula 21) (1.5/1, 2.25 g, 9.9 mmol), p-BBB (2.6 g, 10.4 mmol), and Bu₄NBr (0.32 g, 0.99 mmol) in toluene (25 mL) was added NaOH solution (50 wt %, 1.19 g, 14.9 mmol). The mixture was heated to 75° C. with stirring for 2 h. Additional water (0.9 mL) was added and the mixture was continuously heated at 75° C. for 1 h. HPLC showed the reaction was complete. The mixture was diluted with water (20 mL) and EtOAc (20 mL). The organic layer was washed with water (20 mL), saturated NaCl, and dried over anhydrous MgSO₄. The solvent was removed using a rotoevaporator. The oil was purified by silica gel chromatography eluted with hexane/EtOAc (4/1) to give the compound of Formula 23 (1.92 g, 49%) as a colorless oil (solidified later). ¹H-NMR (CDCl₃) δ 7.70 (s, 2H), 7.31 (d, J=8.3 Hz, 2H), 7.03 (d, J=8.5 Hz, 2H), 4.24 (q, J=7.1 Hz, 4H), 3.94 (s, 2H), 1.19 (t, J=7.3 Hz, 6H). MS (Scan AP+) 396 m/z (M+1, 100%). Calculated for C₁₆H₁₈BrN₃O₄: C, 48.50, H, 4.58, N, 10.60; found: C, 48.52, H, 4.35, N, 10.42. Following evaporation of hexane/EtOAc, the compound of Formula 25 (0.79 g, 20%) was obtained as a white solid: mp=50° C. to 53° C.; ¹H-NMR (CDCl₃) δ 7.87 (s, 1H), 7.62 (s, 1H), 7.29 (d, J=8.3 Hz, 2H), 6.61 (d, J=8.5 Hz, 2H), 4.31 (q, J=7.3 Hz, 4H), 3.89 (s, 2H), 1.28 (s, 6H); MS (Scan AP+) 396 m/z (M+1, 100%); Calculated for C₁₆H₁₈BrN₃O₄: C, 48.50, H, 4.58, N, 10.60; found: C, 48.44, H, 4.29, N, 10.02.

### Example 49

### Purification of 2-(4-bromo-benzyl)-2-[1,2,3]triazol-2-yl-malonic acid diethyl ester (Formula 23) from a mixture of 2-(4-bromo-benzyl)-2-[1,2,3]triazol-2-yl-malonic acid diethyl ester and 2-(4-bromo-benzyl)-2-[1,2,3]triazol-1-yl-malonic acid diethyl ester (Formula 25) via triazolium formation

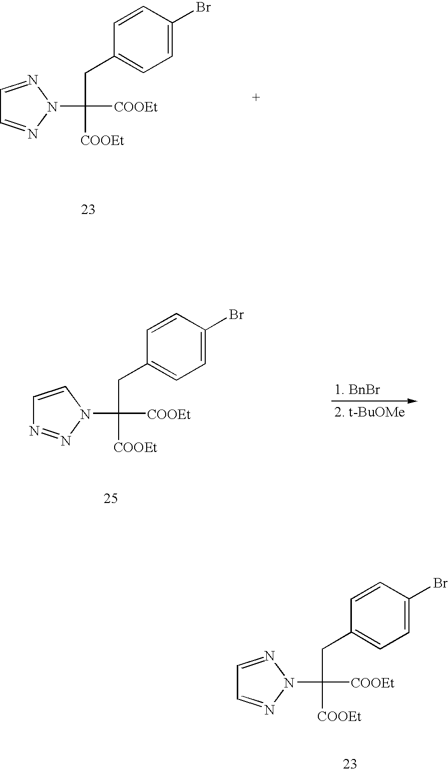

Benzyl bromide (0.37 g, 2.2 mmol) was added to a mixture of 2-(4-bromo-benzyl)-2-[1,2,3]triazol-2-yl-malonic acid diethyl ester (Formula 23) and 2-(4-bromo-benzyl)-2-[1,2,3]triazol-1-yl-malonic acid diethyl ester (Formula 25) (1.5/1, 1.71 g, about 1.7 mmol of Formula 25) and heated at 60° C. for 14 h and then 70° C. for 4 h. Additional BnBr (0.1 g, 0.56 mmol) was added and the mixture was continuously heated at 70° C. for 6 h. The brown reaction solution was diluted with t-BuOMe (30 mL) and heated to reflux for 10 min. After cooling to RT overnight, a suspension was formed with the solid sticking to the bottom of the flask. The top clear solution was decanted and the solvent was removed to give a brown oil (1.02 g, 60% recovery). ¹H-NMR showed the ratio of the compounds of Formula 23 and 25 was 10/1.

### Example 50

### Preparation of 2-(4-bromo-benzyl)-2-[1,2,3]triazol-2-yl-malonic acid diethyl ester (Formula 23)

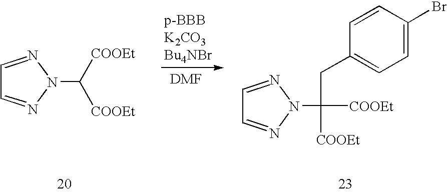

Anhydrous potassium carbonate (100.8 g, 0.73 mole, powder) was added to a solution of 2-[1,2,3]triazol-2-yl-malonic acid diethyl ester (Formula 20) (138 g, 0.608 mole) in dry DMF (400 mL) and stirred for 20 min. The suspension was cooled in a water bath (about 20° C.). To this suspension was added p-BBB (136.7 g, 0.547 mole) followed by Bu₄NBr (19.6 g, 0.061 mole). The water bath was subsequently removed and the reaction was stirred at RT for 20 h. HPLC showed that all of the starting material (Formula 20) had disappeared. The reaction mixture was diluted with t-BuOMe (2 L) and was washed with water (2×1 L), saturated NaCl and dried over anhydrous MgSO₄. The solvent was removed to the compound of Formula 23 as a brown oil (232.4 g, 107%). The product was directly used in Example 51. ¹H-NMR (CDCl₃) δ 7.66 (s, 2H), 7.28 (d, J=8.5 Hz, 2H), 7.00 (d, J=8.8 Hz, 2H), 4.21 (q, J=7.0 Hz, 4H), 3.91 (s, 2H), 1.16 (t, J=7.0 Hz, 6H). MS (Scan AP+) 396 m/z (M+1, 100%). Calculated for C₁₆H₁₈BrN₃O₄: C, 48.5, H, 4.58, N, 10.60; found: C, 48.36, H, 4.61, N, 10.18.

### Example 51

### Preparation of 2-{4-[3-(5-methyl-2-phenyl-oxazol-4-yl)-propyl]-benzyl}-2-[1,2,3]triazol-2-yl-malonic acid diethyl ester (Formula 28)

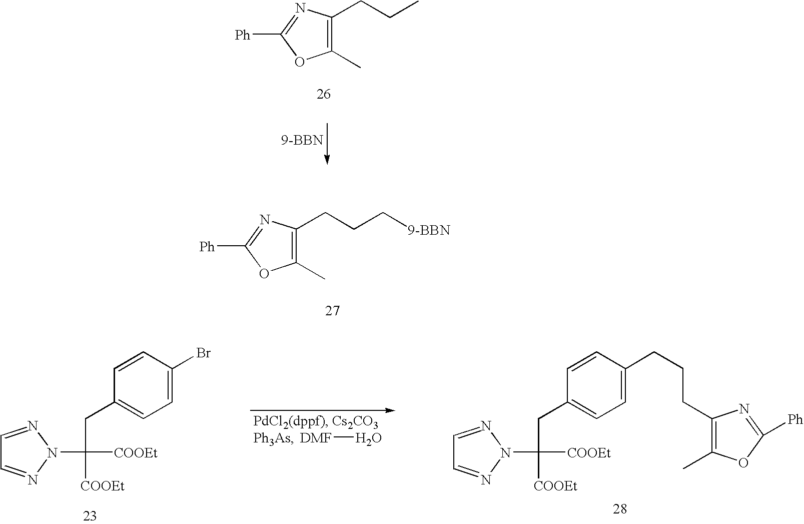

To a 2 L 3-neck round bottom flask purged with nitrogen was added 9-BBN dimer (70.5 g, 0.58 mole; caution: 9-BBN dimer is flammable and may spontaneously combust when exposed to air.) THF (200 mL) was added and the mixture was stirred to give a suspension. A solution of 2-methyl-3-allyl-5-phenyloxazole (109.6 g, 0.55 mole, CAMBRIDGE MAJOR, Lot 205-80-3a, Formula 26) in THF (800 mL) was added under nitrogen. The mixture was stirred at RT under nitrogen for 18 h. Thin layer chromatography (TLC) and ¹H-NMR showed that there was some allyloxazole present. Additional 9-BBN dimer (4.6 g, 0.038 mole) was added and the solution was continuously stirred for 6 h. ¹H-NMR showed only trace amounts of allyloxazole.

In another 3 L 3-neck round bottom flask equipped with a mechanical stirrer, thermometer, and nitrogen inlet, was added 2-(4-bromo-benzyl)-2-[1,2,3]triazol-2-yl-malonic acid diethyl ester (212.4 g, 0.5 mole, Formula 23), PdCl₂(dppf)₂ (8.17 g, 10 mmol), triphenylarsine (6.12 g, 20 mmol), and DMF (1 L). Anhydrous Cs₂CO₃ (195.5 g, 0.6 mole) was added to the mixture with stirring. Nitrogen was then bubbled into the suspension for 10 min. Water (100 mL) was added and nitrogen was continuously bubbled for 20 min. The solution of 9-BBN adduct in THF prepared above was added through PTFE tubing under nitrogen. The suspension was then stirred at RT for 1 h and then at 35° C. for 5 h. Mass spectrometry (MS) showed only a small amount of product had formed. Additional PdCl₂(dppf)₂ (8.17 g, 10 mmol) and triphenylarsine (6.12 g, 20 mmol) was added and the suspension was continuously stirred at 35° C. for 12 h. TLC and MS showed that the reaction was complete.

Reaction solids were removed by filtration and the filter cake was washed with THF (3×150 mL). The filtrate was concentrated in a rotoevaporator to remove most of the THF. The concentrate was diluted with t-BuOMe (3 L) and washed with water (2×1 L). The aqueous layer was back extracted with t-BuOMe (800 mL). The organic layers were combined, washed with saturated NaCl (2×1 L), and dried over anhydrous MgSO₄. The solution was then stirred with activated charcoal (20 g) and heated to reflux for 30 min. After cooling to RT, the charcoal was removed by filtering through CELITE. The filtrate was concentrated to about 500 mL and diluted with hexane (250 mL). The mixture was filtered through a pad of silica gel (180 g silicagel 60, column: 9×5 cm, OD×H, gravity filtration) and washed with t-BuOMe/hexane (1/1, 1 L). The filtrate was concentrated to give crude 2-{4-[3-(5-methyl-2-phenyl-oxazol-4-yl)-propyl]-benzyl}-2-[1,2,3]triazol-2-yl-malonic acid diethyl ester as a brown oil (353.6 g). The product was used directly in Example 52.

### Example 52

### Preparation of (S/R)-3-{4-[3-(5-methyl-2-phenyl-oxazol-4-yl)-propyl]-phenyl}-2-[1,2,3]triazol-2-yl-propionic acid (Formula 29)

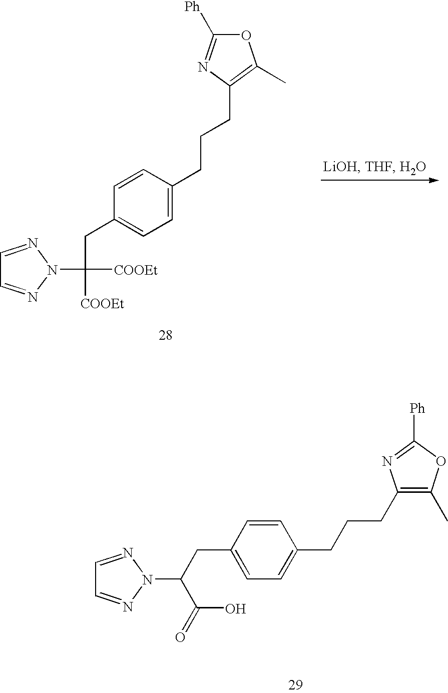

To a solution of crude 2-{4-[3-(5-methyl-2-phenyl-oxazol-4-yl)-propyl]-benzyl}-2-[1,2,3]triazol-2-yl-malonic acid diethyl ester (about 0.5 mole, Formula 28) in THF (1.5 L) was added a solution of LiOH monohydrate (52.5 g, 1.25 mole) in water (500 mL). The reaction temperature rose to 34° C. after the addition and stayed around 30° C. for 1 h. Stirring for 3 h after the addition, HPLC showed about 90% hydrolysis. Additional LiOH monohydrate (10.5 g, 0.25 mole) in water (200 mL) was added and continuously stirred at RT for an additional 16 h. HPLC showed all of the starting material had disappeared. THF was removed using a rotoevaporator to give an orange suspension. Water (2 L) was added and stirred for 20 min. The solid was removed by filtration. The filtrate (3 L) was extracted with EtOAc (2 L, 1.7 L, then 1.2 L). The last extraction was allowed to sit overnight before separation of the two layers. The aqueous layer was subsequently acidified to pH 2 with slow addition of concentrated HCl (120 mL) over a period of 2 h. The suspension was cooled in an ice bath to about 10° C. and stirred for additional 30 min. The solid was collected by filtration, washed with water (2×300 mL), and dried under vacuum to give a yellow solid (176 g). The solid was slurried and heated in ACN (200 mL) for 20 min. After cooling to RT, the solid was collected by filtration. The filter cake was washed with acetonitrile (80 mL) and dried under vacuum to give (S/R)-3-{4-[3-(5-methyl-2-phenyl-oxazol-4-yl)-propyl]-phenyl}-2-[1,2,3]triazol-2-yl-propionic acid as a slightly yellow solid (163 g, 78% yield overall for the 3 steps): ¹H-NMR (DMSO-d6) δ 13.34 (s, 1H), 7.84 (d, J=8.1 Hz, 2H), 7.69 (s, 2H), 7.42 (m, 3H), 6.95 (m, 4H), 5.59 (m, 1H), 3.43 (dd, 2H), 2.46 (m, 2H), 2.34 (t, J=7.3 Hz, 2H), 1.77 (p, J=7.5 Hz, 2H). MS (Scan AP+) 417 m/z (M+1, 100%). Calculated for C₂₄H₂₄N₄O₃: C, 69.21, H, 5.81, N, 13.45; found: C, 68.99, H, 5.69, N, 13.27; Pd contains: 283 ppm.

### Example 53

### (S)-3-{4-[3-(5-methyl-2-phenyl-oxazol-4-yl)-propyl]-phenyl}-2-[1,2,3]triazol-2-yl-propionic acid (Formula 30)

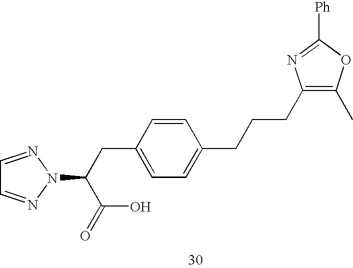

(S/R)-3-{4-[3-(5-methyl-2-phenyl-oxazol-4-yl)-propyl]-phenyl}-2-[1,2,3]triazol-2-yl-propionic acid (100 g, 0.24 mole, Formula 29) was isolated by chiral separation. The chiral separation used a 50×10 cm ID prepacked CHIRALPAK AD (20 μm particles) column and a mobile phase of 75:25:0.1 n-heptane/ETOH/TFA at a rate of 275 mL/min. Following chromatographic separation, TFA in the column eluate was neutralized using 0.2% Et₃N to prevent formation of an ethyl ester of the compound of Formula 30. The desired enantiomer in the column eluate was concentrated using a rotoevaporator under reduced pressure, which provided a crude Et₃N salt of (S)-3-{4-[3-(5-methyl-2-phenyl-oxazol-4-yl)-propyl]-phenyl}-2-[1,2,3]triazol-2-yl-propionic acid as a yellow oil (total 232 g) with chiral purity of 95.5% e.e. The major by-product in the oil was the Et₃N salt of TFA.

The crude Et₃N salt of the compound of Formula 30 was purified in 3 lots (10.79 g, 11.16 g, 209.97 g). The crude Et₃N salt (209.97 g) was diluted with water (1 L) followed by the addition of EtOAc (600 mL). Hydrochloric acid (1 M, about 58 mL) was added slowly with stirring until the pH of the solution reached 3.93. The two layers were separated, and the aqueous layer was back extracted with EtOAc (100 mL). The organic layers were combined and washed with water (120 mL), saturated NaCl, and dried over anhydrous MgSO₄. The solvent was removed on rotoevaporator to give a white foam (46.6 g). ¹H-NMR spectrum showed the disappearance of triethylamine and ¹⁹F-NMR spectrum showed the disappearance of TFA. The white foam was dissolved in ACN (400 mL) with heating. The solution was subsequently allowed to cool to 40° C. in about 2.5 h and was maintained at 40° C. for 3 hours, cooled to 35° C. and maintained at 35° C. for 3 hours, and finally cooled to RT overnight. The solid was collected by filtration, washed with ACN (50 mL) and dried under vacuum to give (S)-3-{4-[3-(5-methyl-2-phenyl-oxazol-4-yl)-propyl]-phenyl}-2-[1,2,3]triazol-2-yl-propionic acid as crystalline white solid (36.29 g). Chiral HPLC showed 100% e.e. Purified material from the 3 lots was combined to give 39.96 g of the titled compound (79.9% recovery). ¹H-NMR (DMSO-d6) δ 13.36 (s, 1H), 7.87 (d, J=8.0 Hz, 2H), 7.71 (s, 2H), 7.44 (m, 3H), 6.98 (m, 4H), 5.61 (m, 1H), 3.45 (dd, 2H), 2.48 (m, 2H), 2.37 (t, J=7.3 Hz, 2H), 1.79 (p, J=7.6 Hz, 2H). MS (Scan AP+) 417 m/z (M+1, 100%). Calculated for C₂₄H₂₄N₄O₃: C, 69.21, H, 5.81, N, 13.45; found: C, 69.46, H, 5.77, N, 13.28; Pd contains: 7 ppm, B contains: 5 ppm, Fe contains: 6 ppm; chiral purity: 99.22% e.e.; percent parent: 99.0%.

The combined mother liquors from the 3 lots were concentrated to give a yellow solid (8.28 g). The solid was slowly recrystallized from ACN (105 mL) as described above. The solid was collected by filtration to give a yellow crystalline solid (3.08 g, 29% e.e. by chiral HPLC). The mother liquor was concentrated to give a yellow solid (5.47 g, 97% e.e. by chiral HPLC).

It should be noted that, as used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated herein by reference in their entirety and for all purposes.

What is claimed is:

1. A method of making a compound of Formula 1, 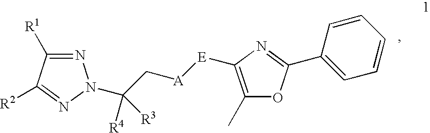
or a pharmaceutically acceptable salt, ester, amide, or prodrug thereof,
wherein R¹ and R² are independently hydrogen, halogen, aryl, benzoyl, C₁₋₆ alkyl, C₁₋₆ haloalkyl, C₁₋₆ alkanoyl, C₁₋₆ haloalkanoyl, or C₃₋₇ cycloalkanoyl;
R³ and R⁴ are electron-withdrawing groups, which may be the same or different;
E is C₁₋₆ alkyleneoxy, C₁₋₆ alkyleneamino, C₁₋₆ alkylenethio, C₁₋₆ alkanediyl, C₁₋₆ alkenediyl, or C₁₋₆ alkyndiyl; and
A is arylene or heteroarylene, each of which may have one or more non-hydrogen substituents, provided that when A is a five-member heteroarylene group, A is not linked to E through a heteroatom, the method comprising:
(a) reacting a [1,2,3]triazole salt of Formula 2, 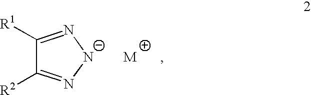
with a compound of Formula 3, 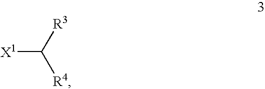
to yield a compound of Formula 4, 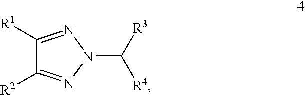
wherein R¹, R², R³, and R⁴ are as defined above for Formula 1, M is a counter ion, and X¹ is a leaving group;
(b) reacting the compound of Formula 4 with a compound of Formula 7, 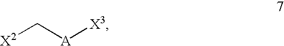
to yield a compound of Formula 8, 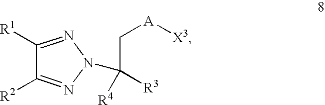
wherein R¹, R², R³, R⁴, and A are as defined above for Formula 1, X² is a leaving group, and X³ is a leaving group or a nucleophilic group, including hydroxy, amino, or thio;
(c) coupling the compound of Formula 8 and a compound of Formula 9, 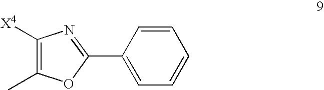
to yield the compound of Formula 1, wherein X⁴ is a C₁₋₆ hydroxyalkyl, C₁₋₆ oxoalkyl, C₁₋₆ haloalkyl, C₂₋₆ alkenyl, or C₂₋₆ alkynyl; and
(d) optionally converting the compound of Formula 1 into a pharmaceutically acceptable salt, ester, amide, or prodrug.

2. The method of claim 1, wherein R¹ and R² in Formula 2 are both hydrogen, M in Formula 2 is a Group 1 or Group 2 metal ion, or R¹ and R² in Formula 2 are both hydrogen and M in Formula 2 is a Group 1 or Group 2 metal ion.

3. The method of claim 1, wherein R³ and R⁴ in Formula 3 are independently cyano, C₁₋₆ alkanoyl, carboxy, C₁₋₆ alkoxycarbonyl, carbamoyl, C₁₋₆ alkylaminocarbonyl, C₁₋₆ dialkylaminocarbonyl, sulfonylaminocarbonyl, C₁₋₆ alkylsulfonylaminocarbonyl, N-C₁₋₆ alkylsulfonyl-N-C₁₋₆ alkylaminocarbonyl, or C₁₋₆ alkylsulfonyl, or R³ and R⁴ together with a carbon to which R³ and R⁴ are attached comprise a β-dicarbonyl moiety.

4. The method of claim 1, further comprising:
hydrolyzing R³ and R⁴ to yield a pair of carboxy groups; and
removing one of the carboxy groups through contacting with an acid, wherein R³ and R⁴ in Formula 3 are both C₁₋₆ alkoxycarbonyl.

5. The method of claim 1, wherein the compound of Formula 3 is a malonic acid dialkyl ester, including a derivative of dimethyl malonate or diethyl malonate, or is a 3-oxo-C₄₋₉ alkanoic acid C₁₋₆ alkyl ester, including ethyl acetoacetate.

6. The method of claim 1, further comprising one of the following:
(a) coupling the compounds of Formula 8 and Formula 9 under Mitsunobu conditions to yield the compound of Formula 1, wherein E in Formula 1 is C₁₋₆ alkyleneoxy, X³ in Formula 8 is hydroxy, and X⁴ in Formula 9 is C₁₋₆ hydroxyalkyl;
(b) coupling the compounds of Formula 8 and Formula 9 in the presence of a base to yield the compound of Formula 1, wherein E in Formula 1 is C₁₋₆ alkyleneoxy or C₁₋₆ alkylenethio, X³ in Formula 8 is hydroxy or thio, and X⁴ in Formula 9 is C₁₋₆ haloalkyl;
(c) reacting the compounds of Formula 8 and Formula 9 in the presence of catalytic amounts of an acid to form an imine intermediate; and
reducing the imine intermediate to yield the compound of Formula 1, wherein E in Formula 1 is C₁₋₆ alkyleneamino, X³ in Formula 8 is amino, and X⁴ in Formula 9 is C₁₋₆ oxoalkyl;
(d) coupling the compounds of Formula 8 and Formula 9 in the presence of an organometallic catalyst to yield the compound of Formula 1, wherein E in Formula 1 is C₁₋₆ alkenediyl or C₁₋₆ alkyndiyl, X³ in Formula 8 is a leaving group, and X⁴ in Formula 9 is C₂₋₆ alkenyl or C₂₋₆ alkynyl; or
(e) reacting the compound of Formula 9 with a hydroboration agent to form an alkyl- or alkenyl-adduct; and
reacting the alkyl- or alkenyl-adduct with the compound of Formula 8 in the presence of a Pd catalyst to produce the compound of Formula 1, wherein E is C₁₋₆ alkanediyl or C₁₋₆ alkenediyl, X³ in Formula 8 is a leaving group, and X⁴ in Formula 9 is C₂₋₆ alkenyl or C₂₋₆ alkynyl.

7. A method of making a compound of Formula 4, 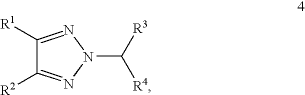
in which R¹ and R² are independently hydrogen, halogen, aryl, benzoyl, C₁₋₆ alkyl, C₁₋₆ haloalkyl, C₁₋₆ alkanoyl, C₁₋₆ haloalkanoyl, or C₃₋₇ cycloalkanoyl; and
R³ and R⁴ are electron-withdrawing groups, which may be the same or different, the method comprising:
reacting a [1,2,3]triazole salt of Formula 2, 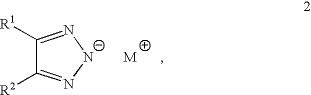
with a compound of Formula 3, 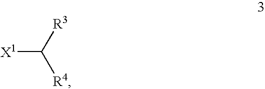
to yield the compound of Formula 4, wherein R¹, R², R³, and R⁴ are as defined above for Formula 4, M is a counter ion, and X¹ is a leaving group.

8. A method of concentrating an N2-alkylated triazole of Formula 4, 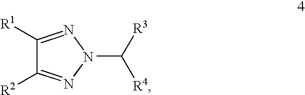
or of Formula 8, 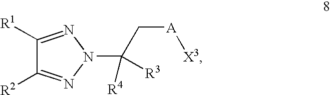
in a mixture of N-alkylated triazoles that includes at least one N1-alkylated triazole of Formula 5, 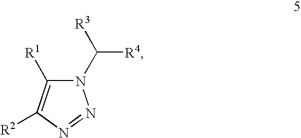
or of Formula 14 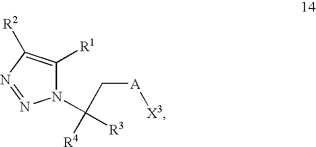
wherein R¹ and R² are independently hydrogen, halogen, aryl, benzoyl, C₁₋₆ alkyl, C₁₋₆ haloalkyl, C₁₋₆ alkanoyl, C₁₋₆ haloalkanoyl, or C₃₋₇ cycloalkanoyl;
R³ and R⁴ are electron-withdrawing groups, which may be the same or different;
A is arylene or heteroarylene, each of which may have one or more non-hydrogen substituents; and
X³ is a leaving group or a nucleophilic group, including hydroxy, amino, or thio, the method comprising:
(a) reacting the mixture of N-alkylated triazoles with an alkylating agent to convert the at least one N1-alkylated triazole to one or more N1,N3-bisalkylated triazolium intermediates;
(b) contacting the one or more N1,N3-bisalkylated triazolium intermediates with a solvent that is adapted to precipitate out of solution the one or more N1,N3-bisalkylated triazolium intermediates while leaving the N2-alkylated triazole in solution; and
(c) optionally filtering out the precipitate.

9. A compound of Formula 4, 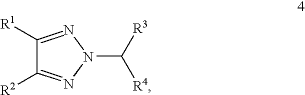
or a compound of Formula 8, 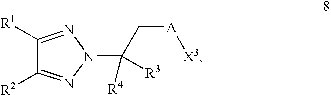
or salts thereof, wherein R¹ and R² are independently hydrogen, halogen, aryl, benzoyl, C₁₋₆ alkyl, C₁₋₆ haloalkyl, C₁₋₆ alkanoyl, C₁₋₆ haloalkanoyl, or C₃₋₇ cycloalkanoyl;
R³ and R⁴ are each an electron-withdrawing group including cyano, C₁₋₆ alkanoyl, carboxy, C₁₋₆ alkoxycarbonyl, carbamoyl, C₁₋₆ alkylaminocarbonyl, C₁₋₆ dialkylaminocarbonyl, sulfonylaminocarbonyl, C₁₋₆ alkylsulfonylaminocarbonyl, N-C₁₋₆ alkylsulfonyl-N-C₁₋₆ alkylaminocarbonyl, or C₁₋₆ alkylsulfonyl, and may be the same or different provided that R³ and R⁴ are not both methoxycarbonyl or ethoxycarbonyl;
A is arylene or heteroarylene, each of which may have one or more non-hydrogen substituents; and
X³ is a leaving group or a nucleophilic group, including hydroxy, amino, or thio.

10. The compounds of claim 9 in which R³ and R⁴ together with the carbon to which R³ and R⁴ are attached comprise a β-dicarbonyl moiety.
